(19) United States
(12) Patent Application Publication
(10) Pub. No.: US 2018/0346531 A1
(43) Pub. Date: Dec. 6, 2018

(54) COMPOSITIONS AND METHODS FOR DELIVERING BIOTHERAPEUTICS

(71) Applicant: Regents of the University of California, Oakland, CA (US)

(72) Inventor: Benjamin Pyles, Woodland, CA (US)

(73) Assignee: Regents of the University of California, Oakland, CA (US)

(21) Appl. No.: 15/759,788

(22) PCT Filed: Aug. 23, 2016
(86) PCT No.: PCT/US2016/048122

§ 371 Date: Mar. 13, 2018

Related U.S. Application Data
(60) Provisional application No. 62/219,031, filed on Sep. 15, 2015.

(51) Int. Cl. C07K 14/47; A61K 49/00; C07K 14/00; A61P 25/00

(52) U.S. Cl. CPC C07K 14/4703; A61K 49/0017; C07K 14/00; A61P 25/00; C07K 2319/09; A61K 38/00; C07K 2319/35; C07K 2319/60; C07K 2319/42; C07K 2319/81; C07K 2319/24

(57) ABSTRACT

Provided herein are compositions and methods relating to cell-penetrating conjugates for the delivery of therapeutic polypeptides or polynucleotides to cells or tissues of the body. The delivery conjugate comprises a cell-penetrating peptide and a nuclear localization signal sequence plus an effector moiety (such as a polypeptide or polynucleotide) as the payload, optionally further including an epitope tag as well as a solubility peptide and a configurating peptide. The delivery conjugate can also include a component capable of specifically directing the conjugate to a target cell or tissue, making the conjugate effective for treating diseases in the target tissue.

### RELATED APPLICATIONS

This application claims priority to U.S. Provisional Patent Application No. 62/219,031, filed Sep. 15, 2015, the contents of which are hereby incorporated by reference in the entirety for all purposes.

### STATEMENT AS TO RIGHTS TO INVENTIONS MADE UNDER FEDERALLY SPONSORED RESEARCH AND DEVELOPMENT

This invention was made with government support under Grant No. NS071028 awarded by the National Institutes of Health. The Government has certain rights in the invention.

### BACKGROUND OF THE INVENTION

Protein-based and gene-based therapies have great potential for the treatment and/or prevention of numerous diseases such as inherited and acquired disease. Multiple barriers impede the delivery of therapeutic biomolecules, e.g., polypeptides and polynucleotides to specific target cells and tissues in vivo. For instance, current delivery methods are unable to effectively deliver therapeutic biomolecules to the brain or other target tissues. Liposomes and other nanoparticles cannot provide universal or systemic delivery. Viral vectors also have limited biodistribution and must be physically placed into the cells or tissue of interest. There remains a need in the field for an effective delivery system that can transport a therapeutic biomolecule to a target cell and/or tissue in a subject having a disease or disorder that can be treated by the therapeutic biomolecule.

### BRIEF SUMMARY OF THE INVENTION

In a first aspect, provided herein is a delivery conjugate comprising (1) a cell penetrating peptide, a solubilizing peptide and a configuring peptide; and (2) an effector. The cell penetrating peptide can be selected from a group consisting of a HIV TAT protein transduction domain peptide, Drosophila Antennapedia (Antp) peptide and polyarginine (Arg₈) peptide. The solubilizing peptide can be a maltose-binding protein (MBP) peptide. The configuring peptide can be a fluorophore. In some embodiments, the fluorophore is derived from Discosoma sp. The cell penetrating peptide, solubilizing peptide, and configuring peptide can be linked sequentially by one or more peptide bonds. In some embodiments, the cell penetrating peptide (A), solubilizing peptide (B), and configuring peptide (C) are ordered starting at the amino-terminus as ABC and ACB. The effector is a molecule of any chemical nature with a predetermined desirable biological activity and is to be delivered to a target organ or tissue for therapeutic purposes. In some instances, the effector is a polypeptide or a polynucleotide.

In some embodiments, the effector of the delivery conjugate is a polypeptide. The delivery conjugate may be a fusion protein wherein the cell penetrating peptide, solubilizing peptide and configuring peptide; and the effector are linked by a peptide bond. In some embodiments, the delivery conjugate also includes a cell targeting signal.

In other embodiments, the effector is a polynucleotide. The delivery conjugate can further comprise a nuclear localization signal (NLS) located between (1) the cell penetrating peptide, solubilizing peptide and configuring peptide and (2) the effector, e.g., a polynucleotide. In some embodiments, the delivery conjugate also include a cell targeting signal.

In a related aspect, the delivery conjugate comprises these key components: (1) a cell penetrating peptide; (2) a nuclear localization signal sequence; and (3) an effector moiety. The cell penetrating peptide is capable of directing the conjugate to enter into a cell especially a mammalian cell such as a human cell. An HIV TAT protein contains a protein transduction domain, which is characterized as YGRKKRRQRRR or GRKKRRQRRR (see, e.g., Frankel and Pabo, Cell 55:1189-1193, 1988; Green and Loewenstein, Cell 55:1179-1188, 1988; and Vives et al., J. Bio. Chem. 272:16010-16017, 1997) and allows the TAT protein to enter cells by crossing the cell membrane. Thus, an HIV TAT protein or a fragment thereof comprising YGRKKRRQRRR or GRKKRRQRRR can serve as such a cell penetrating peptide in the delivery conjugate of this invention. On the other hand, a nuclear localization signal sequence is a peptide sequence that facilitates translocation of the conjugate into the cell nucleus. One example of a nuclear localization signal is one derived from the SV40 large T antigen, characterizes as PKKKRKVEDPYC, see, e.g., Schirmbeck et al., J. Mol. Med. 79:343, 2001; Vaysse et al., J. Biol. Chem. 279:5555, 2004.

In addition to the three key components of a cell penetrating peptide, a nuclear localization signal sequence, and an effector moiety, the delivery conjugate may further includes the component of an epitope tag. For example, an human influenza hemagglutinin (HA) epitope tag, corresponding to amino acids 98-106 of the HA molecule, is useful for this purpose. While such epitope tag typically does not interfere with the conjugate's biological activity, it facilitates the detection, isolation, and purification of the conjugate. An exemplary sequence of the HA epitope tag is YPYDVPDYA.

In other alternative embodiments, the conjugate may in addition include a solubilizing peptide and/or a configurating peptide. A solubilizing peptide, such as a maltose binding protein, increases the overall solubility of the conjugate and therefore enhances the effectiveness of the delivery conjugate. A configurating peptide, such as fluorophore mCherry, facilitates the delivery of conjugate by maintaining a favorable 3-dimensional configuration of the conjugate. Useful configurating peptides include mCherry and the majority of red fluorescent proteins, which are derived from a protein isolated from Discosoma sp., and other fluorescent proteins in the green range, which are often variants of green fluorescent proteins from Aequorea Victoria.

In a second aspect, provided herein is a method of delivering an effector into a subject in need thereof. The method includes administering to the subject the delivery conjugate disclosed herein. In some embodiments, the subject suffers from a disease involving a genetic locus that is imprinted, dominant, or haploinsufficient or a disease which can be treated by activation or repression of a gene. In some instances, the disease is selected from the group consisting of Angelman Syndrome, Prader-Willi Syndrome, Rett Syndrome, Huntington Disease, 22q11.2 deletion Syndrome, Pitt-Hopkins Syndrome, autism spectrum disorder, depression, schizophrenia, and HIV/AIDS. The step of administering can include administering the delivery conjugate intraperitoneally, subcutaneously, intravenously, or intracranially.

In a third aspect, provided herein is a polynucleotide sequence encoding a cell penetrating peptide, a solubilizing peptide, and a configuring peptide. The cell penetrating peptide, solubilizing peptide, and configuring peptide can be linked sequentially by one or more peptide bonds. In some embodiments, the cell penetrating peptide is selected from a group consisting of a HIV TAT protein transduction domain peptide, Drosophila Antennapedia (Antp) peptide and polyarginine (Arg8) peptide. In some embodiments, the solubilizing peptide is a MBP peptide. In some embodiments, the configuring peptide is a fluorophore. The polynucleotide sequence can also include a coding sequence encoding an effector polypeptide. Optionally, the polynucleotide sequence can contain a coding sequence encoding a nuclear localization signal.

In a fourth aspect, provided herein is an expression cassette comprising a promoter operably linked to the polynucleotide sequence encoding a cell penetrating peptide, a solubilizing peptide, and a configuring peptide. In some cases, the cell penetrating peptide, solubilizing peptide, and configuring peptide are linked sequentially by one or more peptide bonds. Also provided herein is a host cell comprising the expression cassette described herein.

Other objects, features, and advantages of the present invention will be apparent to one of skill in the art from the following detailed description and figures.

### BRIEF DESCRIPTION OF THE DRAWINGS

FIGS. 1A-IC provide the ATF targeting strategy and binding results. FIG. 1A shows ATFs in relationship to the genomic region on the mouse chromosome 7. Imprinting in this region results in genes with paternal-only (blue), maternal-only (pink), or silenced (gray) expression. Genes outside this region are biallelicly expressed (yellow). In the Angelman Syndrome mouse model used in these studies, a targeted insertion with a stop codon replaces exon five of the maternal Ube3a (red X). U-exons, upstream exons of the Ube3a-ATS; PWS-IC, Prader-Willi Syndrome imprinting control region either methylated (filled circle) or unmethylated (open circle). Luciferase assays of 12 ATF in HEK293T cells. Bars indicate log₂ transformed fold activation or repression of luciferase in the presence of the ATF compared to an empty vector control. Error bars indicate standard deviation. *, P<0.05; two-tailed homoscedastic t-test, representing three biological replicates. FIG. 1B shows representative EMSA of S1 zinc finger array binding its cognate target. FIG. 1C shows ChIP-PCR data for the HA-tagged ATF S1 or empty vector (EV) at the S1 chromosomal target site in mouse Neuro2A cells. IgG serves as a locus and negative control, respectively. ChIP-enrichment is shown relative to 0.1% chromatin input.

FIGS. 2A-2E show the distribution of TAT-S1 and TAT-R6 in adult mouse. FIG. 2A illustrates the structure of TAT-S1 and TAT-R6 ATF proteins. MBP, maltose binding protein; ZF, zinc finger. FIG. 2B shows mCherry fluorescence/ambient light merged image of live wild type C57BL/6 mice 4 h post injection with TAT-S1, ATF injection buffer (Mock), or the negative control TAT-R6 (0.16-0.20 g/kg, IP). Fur was shaved to improve the fluorescent signal. FIG. 2C shows kinetics of fluorescence in the brain for TAT-S1 (green triangles), Mock (red squares), and TAT-R6 (blue diamonds). *, p<0.005 compared to Mock at that timepoint, two-tailed homoscedastic t-test, n=5). FIG. 2D shows mice harvested after 4 h. Skin on the back was removed to improve the fluorescent signal. Intense signal can be seen in the kidneys. FIG. 2E shows internal organs harvested after 4 h. Note that the bright white kidney in the TAT-R6-injected sample indicates florescence in excess of the maximum setting.

FIGS. 3A-3F show reactivation of Ube3a in a mouse model of Angelman Syndrome by TAT-S1 but not TAT-R6. FIG. 3A shows ATFs that were injected (160-200 mg/kg, subcutaneous [SC]) three times per week for four weeks, with the final inject four hours before harvest. FIG. 3B shows TAT-S1 distribution in a whole brain sagittal section (α-HA, 5 μm) from wild type mice receiving no treatment (NT) or TAT-S1. White dashed line indicates outline of brain section. FIG. 3C provides imaging of Ube3a protein expression (green) or DAPI (blue) in brain slices of the hippocampus and cerebellum (α-Ube3a [Sigma E8655], 50 μm). Sections from no treatment (NT) wild type and AS mice are shown as controls. A 10% linear brightness reduction was applied equally to the green channel of all images to reduce autoflorescence and clarify features. FIG. 3D provides quantification of Ube3a from unaltered images of the same regions in different mice. AFI, average fluorescence intensity; *, p<0.01, one-sided Welch's t-test for unequal variance, n=3-4 mice. FIG. 3E provides western blot of Ube3a from brain cytosolic lysates of three different mice that received the indicated treatment (α-Ube3a, Sigma E8655). Ponceau S staining of the membrane is shown as a loading control. FIG. 3F shows results of two rotarod experiments testing non-treated AS and WT mice (left), and TAT-S1-treated AS and Mock-treated WT mice (right). *, p<7.6×10⁻⁵, two-sided homoscedastic Student's t-test, n=16-19 mice; ns, not significant. Error bars indicate standard error.

FIGS. 4A-4E provide the sequences of proteins and reporters used in this study.

FIGS. 5A-5B show TAT-ATFs before and after injection into mice. FIG. 5A shows a Coomassie-stained gel demonstrating the purity of three separate preparations of TAT-S1 and TAT-R6 protein. Filled arrow, 100 kD full-length protein band is actually two close bands representing proteins with and without an attached MBP domain. Open arrow, 44 kD maltose binding protein. FIG. 5B shows a Western blot detecting the HA-tag in brain nuclear lysate 4 hours post injection. Filled arrow, 100 kD full-length protein band. Lower molecular weight breakdown products containing the HA tag are also visible. S1, TAT-S1; R6, TAT-R6; M, mock injection; NT, no treatment.

FIGS. 6A-6B show high resolution images of Ube3a activation by TAT-S1 but not TAT-R6. A mouse model of Angelman Syndrome (AS) was injected with TAT-S1 or TAT-R6 (160-200 mg/kg, intraperitoneal [IP]) three times per week for 7.5 weeks. FIG. 6A shows high-resolution imaging of Ube3a protein expression in brain slices of the hippocampus hilus region of the dentate gyrus and the cerebellum Purkinje cell layer (α-Ube3a [Atlas HPA039410], 5 μm). Sections from no treatment (NT) wild type and AS mice are shown as controls. All images are unaltered, with no adjustment for autofluoresence. FIG. 6B shows quantification of Ube3a from unaltered images of the same regions in different mice. AFI, average fluorescence intensity; *, p<0.01, one-sided Welch's t-test for unequal variance, n=3-4 mice.

### DETAILED DESCRIPTION OF THE INVENTION

### I. Introduction

The present disclosure is based, in part, upon the surprising discovery of a delivery conjugate containing a cell penetrating peptide, solubilizing peptide and configuring peptide linked to a therapeutic effector moiety such as a polypeptide or polynucleotide effectively enters a target cell to transport the payload into the cell or tissue. The novel combination of a cell penetrating peptide, a solubilizing peptide and a configuring peptide into a delivery conjugate stabilizes the conjugate within the body and enables the internalization of the therapeutic payload into a cell or tissue. As illustrated in Example 1, a delivery conjugate as described herein can be administered to a subject by injection and can disperse to various tissues to the body, and even cross the blood-brain barrier. Also provided herein is a polynucleotide sequence encoding the delivery conjugate, an expression cassette containing the polynucleotide sequence operably linked to the polynucleotide sequence, and a host cell comprising the expression cassette disclosed herein.

### II. Definitions

Unless defined otherwise, technical and scientific terms used herein have the same meaning as commonly understood by a person of ordinary skill in the art. See, e.g., Lackie, Dictionary of Cell and Molecular Biology, Elsevier (4.sup.th ed. 2007); Sambrook et al., Molecular Cloning, A Laboratory Manual, Cold Springs Harbor Press (Cold Springs Harbor, N.Y. 1989). Any methods, devices and materials similar or equivalent to those described herein can be used in the practice of this invention. For purposes of the present invention, the following terms are defined.

The terms “a,” “an,” or “the” as used herein not only include aspects with one member, but also include aspects with more than one member. For instance, the singular forms “a,” “an,” and “the” include plural referents unless the context clearly dictates otherwise. Thus, for example, reference to “a cell” includes a plurality of such cells and reference to “the agent” includes reference to one or more agents known to those skilled in the art, and so forth.

The term “cell penetrating peptide” refers to a peptide that can introduce a polypeptide or polynucleotide into the cytoplasm or nucleus of a cell.

The term “effector,” “cargo molecule,” or “payload” is used interchangeably refer to a polypeptide, polynucleotide, molecule, small molecule, chemical compound, or material to be transported into the cell.

The terms “nucleic acid,” “oligonucleotide,” “polynucleotide, and like terms refer to deoxyribonucleotides or ribonucleotides and polymers thereof in either single- or double-stranded form, and complements thereof. These terms are not to be construed as limiting with respect to the length of a polymer. The terms encompass nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphorothioates, phosphoramidates, methyl phosphonates, chiral-methyl phosphonates, 2-O-methyl ribonucleotides, peptide-nucleic acids (PNAs).

Unless otherwise indicated, a particular nucleic acid sequence also implicitly encompasses conservatively modified variants thereof (e.g., degenerate codon substitutions) and complementary sequences, as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); Rossolini et al., Mol. Cell. Probes 8:91-98 (1994)).

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

The term “conservatively modified variants” applies to both amino acid and nucleic acid sequences. With respect to particular nucleic acid sequences, conservatively modified variants refers to those nucleic acids which encode identical or essentially identical amino acid sequences, or where the nucleic acid does not encode an amino acid sequence, to essentially identical sequences. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid which encodes a polypeptide is implicit in each described sequence with respect to the expression product, but not with respect to actual probe sequences.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the invention.

The following eight groups each contain amino acids that are conservative substitutions for one another: 1) Alanine (A), Glycine (G); 2) Aspartic acid (D), Glutamic acid (E); 3) Asparagine (N), Glutamine (Q): 4) Arginine (R), Lysine (K); 5) Isoleucine (I), Leucine (L), Methionine (M), Valine (V); 6) Phenylalanine (F), Tyrosine (Y), Tryptophan (W); 7) Serine (S), Threonine (T): and 8) Cysteine (C), Methionine (M) (see, e.g., Creighton, Proteins (1984)).

The term “polypeptide,” “protein” and “peptide” as used herein can be used interchangeably and refer to the polymer of amino acids. The polypeptide, protein or peptide as described herein may contain naturally-occurring amino acids, as well as non-naturally-occurring amino acids, or analogues and simulants of amino acids. For example, the term encompasses amino acid polymers in which one or more amino acid residue is an artificial chemical mimetic of a corresponding naturally occurring amino acid, as well as to naturally occurring amino acid polymers and non-naturally occurring amino acid polymer. The polypeptide, protein or peptide can be obtained by any method well-known in the art, for example, but not limited to, isolation and purification from natural materials, recombinant expression, chemical synthesis, etc.

A polynucleotide or polypeptide sequence is “heterologous” to an organism or a second polynucleotide sequence if it originates from a foreign species, or, if from the same species, is modified from its original form. For example, when a heterologous promoter is said to be operably linked to a coding sequence, it means that the coding sequence is derived from one species whereas the promoter sequence is derived from another, different species; or, if both are derived from the same species, the coding sequence is not naturally associated with the promoter (e.g., the promoter is a genetically engineered promoter or promoter fragment not found naturally associated with the coding sequence).

Nucleic acid or amino acid sequences are “operably linked” (or “operatively linked”) when placed into a functional relationship with one another. For instance, a promoter or enhancer is operably linked to a coding sequence if it regulates, or contributes to the modulation of, the transcription of the coding sequence. Operably linked DNA sequences are typically contiguous, and operably linked amino acid sequences are typically contiguous and in the same reading frame. However, since enhancers generally function when separated from the promoter by up to several kilobases or more and intronic sequences may be of variable lengths, some polynucleotide elements may be operably linked but not contiguous. Similarly, certain amino acid sequences that are non-contiguous in a primary polypeptide sequence may nonetheless be operably linked due to, for example folding of a polypeptide chain.

The term “linked by a peptide bond” refers to presence of a chemical bond formed between two molecules when the carboxyl group of a first molecule reacts with the amino group of a second molecule to release water.

The term “nuclear localization signal” refers to an amino acid sequence located on a protein that is imported into the nucleus of a cell via nuclear transport.

The terms “administer,” “administering,” “administered” or “administration” refer to providing a pharmaceutical composition of a delivery conjugate (e.g., one described herein), to a subject or patient, such as a human subject or patient.

The term “treating” or “treatment” refers to an act that leads to the elimination, reduction, alleviation, reversal, or prevention or delay of onset or recurrence of any symptom of a relevant condition. In other words, “treating” a condition encompasses both therapeutic and prophylactic intervention against the condition.

The term “promoter,” as used herein, refers to a polynucleotide sequence capable of driving transcription of a coding sequence in a cell. Thus, promoters used in the polynucleotide constructs of the invention include cis-acting transcriptional control elements and regulatory sequences that are involved in regulating or modulating the timing and/or rate of transcription of a gene. For example, a promoter can be a cis-acting transcriptional control element, including an enhancer, a promoter, a transcription terminator, an origin of replication, a chromosomal integration sequence, 5′ and 3′ untranslated regions, or an intronic sequence, which are involved in transcriptional regulation. These cis-acting sequences typically interact with proteins or other biomolecules to carry out (turn on/off, regulate, modulate, etc.) gene transcription. A “constitutive promoter” is one that is capable of initiating transcription in nearly all tissue types, whereas a “tissue-specific promoter” initiates transcription only in one or a few particular tissue types. An “inducible promoter” is one that initiates transcription only under particular environmental conditions or developmental conditions.

Nucleic acid or amino acid sequences are “operably linked” (or “operatively linked”) when placed into a functional relationship with one another. For instance, a promoter or enhancer is operably linked to a coding sequence if it regulates, or contributes to the modulation of, the transcription of the coding sequence. Operably linked DNA sequences are typically contiguous, and operably linked amino acid sequences are typically contiguous and in the same reading frame. However, since enhancers generally function when separated from the promoter by up to several kilobases or more and intronic sequences may be of variable lengths, some polynucleotide elements may be operably linked but not contiguous. Similarly, certain amino acid sequences that are non-contiguous in a primary polypeptide sequence may nonetheless be operably linked due to, for example folding of a polypeptide chain.

The term “expression cassette” refers to a nucleic acid construct, generated recombinantly or synthetically, with a series of specified nucleic acid elements that permit transcription of a particular polynucleotide sequence in a host cell. An expression cassette may be part of a plasmid, viral genome, or nucleic acid fragment. Typically, an expression cassette includes a polynucleotide to be transcribed, operably linked to a promoter. Other elements that may be present in an expression cassette include those that enhance transcription (e.g., enhancers) and terminate transcription (e.g., terminators), as well as those that confer certain binding affinity or antigenicity to the recombinant protein produced from the expression cassette.

The term “host cell,” “host cell line,” and “host cell culture” are used interchangeably and refer to cells into which exogenous nucleic acid has been introduced, including the progeny of such cells. Host cells include the primary cell (parent cell) to which the exogenous nucleic acid has been introduced and progeny derived therefrom without regard to the number of passages. Progeny may not be completely identical in nucleic acid content to a parent cell, but may contain mutations.

The terms “target cell” and “target tissue” refer to a cell or tissue, respectively, into which a polypeptide or polynucleotide is delivered by the delivery conjugate described herein.

The terms “target cell” and “target tissue” include a cell of an organ or tissue of a live animal or human, a microorganism found in or on a live animal or human, as well as ex vivo cells or tissues that are derived, harvested, and/or cultured from an animal cell, human cell or microorganism.

The term “individual,” “subject,” or “patient” refers to animals such as mammals, including, but not limited to, primates (e.g., humans), cows, sheep, goats, horses, dogs, cats, rabbits, rats, mice and the like. In some embodiments, the subject is a human. Subjects in need of treatment include patients already suffering from a disease or disorder as well as those prone to developing the disorder.

The term “subject in need of treatment” as used herein, includes individuals who seek medical attention due to risk of, or actual suffering from, a disease or disorder. Subjects also include individuals currently undergoing therapy that seek manipulation of the therapeutic regimen. Subjects or individuals in need of treatment include those that demonstrate symptoms of a disease or are at risk of suffering from a disease or its symptoms. For example, a subject in need of treatment includes individuals with a genetic predisposition or family history for a specific disease, those that have suffered relevant symptoms in the past, those that have been exposed to a triggering substance or event, as well as those suffering from chronic or acute symptoms of the condition. A “subject in need of treatment” may be at any age of life.

In some embodiments, the term “consisting essentially of” refers to a composition in a formulation whose only active ingredient is the indicated active ingredient, however, other compounds may be included which are for stabilizing, preserving, etc. the formulation, but are not involved directly in the therapeutic effect of the indicated active ingredient. In some embodiments, the term “consisting essentially of” can refer to compositions which contain the active ingredient and components which facilitate the release of the active ingredient. For example the composition can contain one or more components that provide extended release of the active ingredient over time to the subject. In some embodiments, the term “consisting” refers to a composition, which contains the active ingredient and a pharmaceutically acceptable carrier or excipient.

The term “therapeutically effective amount or dose” or “therapeutically sufficient amount or dose” or “effective or sufficient amount or dose” refer to a dose that produces therapeutic effects for which it is administered. The exact dose will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins).

The term “administering” refers to oral administration, administration as a suppository, topical contact, intravenous, intraperitoneal, intramuscular, intralesional, intrathecal, intranasal or subcutaneous administration, or the implantation of a slow-release device, e.g., a mini-osmotic pump, to a subject. Administration is by any route, including parenteral and transmucosal (e.g., buccal, sublingual, palatal, gingival, nasal, vaginal, rectal, or transdermal). Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, transdermal patches, etc. One skilled in the art will know of additional methods for administering a therapeutically effective amount of a compound of the present invention for preventing or relieving one or more symptoms associated with a disease.

### III. Detailed Description

A. Delivery Conjugate

The delivery conjugate described herein includes a cell penetrating peptide, a solubilizing peptide, and a configuring peptide which assist in the trafficking of a therapeutic payload of the conjugate into a target cell or a target tissue. In some embodiments, the delivery conjugate is transported into an intracellular compartment of the cell. The components of a delivery conjugate can be joined or linked together by one or more peptide bonds.

The peptides used in the conjugates of the invention can also be functional variants of the peptides as described herein, including peptides that possess at least about 50%, 55%, 60%, 65%, 70%, 75%, 80%, 85%, 90%, 95%, or more sequence identity with the peptides described herein. In certain instances, the peptides can comprise naturally-occurring amino acids and/or unnatural amino acids. The peptides can be natural or synthetic peptides.

1. Cell Penetrating Peptides

A cell penetrating peptide can be a protein transduction domain, and in some embodiment, have a polycationic sequence. Such peptides can introduce therapeutic payloads into a cell without requiring a specific receptor. A cell penetrating peptide can facilitate the uptake of a therapeutic payload into a cell without disrupting the membranes of the cell such as the extracellular membrane, nuclear membrane, mitochondrial membrane, Golgi membrane, endosomal membrane, peroxisomal membrane, lysosomal membrane, and phospholipid membrane. A peptide can, in some embodiments, direct the transport of a payload across a physiological barrier of the body, such as a blood-brain barrier, transmucosal barrier, hematoretinal barrier, skin barrier, gastrointestinal barrier and pulmonary barrier.

A cell penetrating peptide can have basic residues such as lysine and/or arginine. Such a peptide can also be positively charged.

In some embodiments, the cell penetrating peptide is less than 100 amino acids in length, e.g., 99, 90, 95, 85, 80, 75, 70, 65, 60, 55, 50, 49, 48, 47, 46, 45, 44, 43, 42, 41, 40, 39, 38, 37, 35, 35, 34, 33, 32, 31, 30, 29, 28, 27, 26, 25, 24, 23, 22, 21, 20, 19, 17, 16, 15, 14, 13, 12, 11, 10, 9, 8, 7, 6, 5, 4, 3, or less amino acids in length. In some instances, the cell penetrating peptide has a length of 20 amino acids or less, e.g., 20 aa, 19 aa, 18 aa, 17 aa, 16 aa, 15 aa, 14 aa, 13 aa, 12 aa, 11 aa, 10 aa, 9 aa, 8 aa, 7 aa, 6 aa, 5 aa, 4 aa, 3 aa, or 2 aa.

The cell penetrating peptide can include a membrane-interacting peptide, amphiphatic peptide, polyarginine-based peptide, polylysine-based peptide, calcitonic-derived peptide, and the like. Examples of cell penetrating peptides include a Drosophila Antennapedia (Antp) peptide, a penetratin homeodomain derived peptide, an HIV-1 Tat peptide, a VP22 protein from the Herpes Simplex Virus type-1, a protegrin 1 (PG-1) anti-microbial SynB peptide, a chimeric peptide such as transportan, a synthetic polylysine peptide such as a (Lys)₉ peptide and a synthetic polyarginine peptide such as an (Arg)_{A} peptide.

The HIV-1 Tat polypeptide sequence is set forth in, e.g., NCBI RefSeq No. NP_057853. The complete genome of HIV-1 is set forth in, e.g., NCBI RefSeq No. NC_001802. In some embodiments, the Tat peptide described herein includes an amino acid sequence of residues 48-60 of the HIV-1 Tat protein or a fragment thereof. The Tat peptide can have 4 to 12 amino acids, e.g., 4, 5, 6, 7, 8, 9, 10, 11, or 12 amino acids in length. In some embodiments, the Tat peptide contains one or more amino acid types selected from the group consisting of lysine and arginine. In other embodiments, the Tat peptide includes the following sequence: Arg-Lys-Lys-Arg-Arg-Gln-Arg-Arg-Arg (SEQ ID NO: 91) or Arg-Lys-Lys-Arg-Arg-Gln-Arg-Arg-Arg-Gly (SEQ ID NO: 92).

A Drosophila Antennapedia (AntP) peptide can include the homeodomain of the Antennapedia polypeptide, fragments thereof, or variants thereof. The Drosophila Antennapedia polypeptide sequence is set forth in, e.g., GenBank Accession Nos. AAA70214 and AAA70216, and NCBI RefSeq Nos. NP_996167, NP_996168, NP_996170NP_996175 and NP_996176. The Drosophila Antennapedia mRNA (coding) sequence is set forth in, e.g., GenBank Accession Nos. M20704 and M20705, and NCBI RefSeq Nos. NM_206445, NM_206446, NM_206448, NM_206453 and NM_2064454. In some embodiments, the Antp peptide comprises a helix 3 sequence of the Antp polypeptide, or a variant thereof. The cell penetrating peptide can include a peptide having an amino acid sequence of residues 339-355 of Drosophila AntP protein or residues 43-58 of the homeodomain of the Drosophila AntP protein. In some embodiments, the Antp peptide described herein has at least 80% sequence identity to a Drosophila Antp peptide, for example, over the same length. The Antp peptide can be a natural or synthetic peptide.

In some embodiments, the amphiphatic peptide includes any peptide containing a hydrophilic domain and a hydrophobic domain, or similarly, a polar domain and a non-polar domain. The amphiphatic peptide can have sequential assembly of a domain with hydrophobic amino acid residues and a domain with hydrophilic amino acid residues. Alternatively, such a peptide has a conformational structure that positions the hydrophobic residues opposite the hydrophilic residues.

Chemical groups can be covalently attached to the cell penetrating peptide in order to, for example, improve its overall stability in vivo or in vitro, increase it bioavailability, enhance its biological activity, increase its ability to deliver a payload into a cell or tissue such as a target cell or target tissue, and provide additional properties.

2. Solubilizing Peptides

The solubilizing peptide of the delivery conjugate can increase the solubility of the conjugate and facilitate the transport or shuttling of the delivery conjugate including the therapeutic payload across a membrane of a cell. Examples of useful solubilizing peptides include maltose-binding protein (MBP) peptide, thioredoxin (TRX), transcription pausing factor L (NusA), thiol-disulfide oxidoreductase, and glutathione S-transferase (GST).

The maltose binding protein (MBP) is a 40.70 kDa, 370 amino acid, periplasmic protein of E. coli 12, involved in binding and transport of maltose and is encoded by the malE gene. For the production of recombinant proteins in bacteria, MBP can be fused at the amino- or carboxy-terminus of the recombinant protein of interest to affect protein folding and solubility in protein purification methods (Sachdev et al., Methods Enzymol, 2000, 326:312). The E. coli maltose-binding polypeptide sequence is set forth in, e.g., NCBI RefSeq No. NP_418458. The E. coli strain K-12, substrain MG1655 genome sequence is set forth in, e.g., NCBI RefSeq No. NC_000913. In some embodiments, the MBP peptide of the delivery conjugate contains the full-length MPB protein or a fragment thereof. In some embodiments, a fragment of the full-length maltose binding protein is used as a MBP peptide in the present invention. For example, the MBP peptide can be less than 370 amino acids in length, e.g., 360 aa, 350 aa, 340 aa, 330 aa, 320 aa, 310 aa, 300 aa, 290 aa, 280 aa, 270 aa, 260 aa, 250 aa, 240 aa, 230 aa, 220 aa, 210 aa, 200 aa, 190 aa, 180 aa, 170 aa, 160 aa, 150 aa, 140 aa, 130 aa, 120 aa, 110 aa, 100 aa, 90 aa, 80 aa, 70 aa, 60 aa, 50 aa, 40 aa, 30 aa, 20 aa, 10 aa or less in length.

3. Configurating Peptides

Without being bound to a particular theory, the configuring peptide can facilitate in the systemic delivery of payload-containing conjugate through the body of a subject by maintaining a favorable 3-dimensional configuration of the conjugate. Examples of useful configuring peptides include fluorophores derived from a fluorescent protein isolated from Discosoma sp. such as mCherry, mRFP1, DsRed, mTomato, tdTomato, mApple, mKO2, mKate, mRuby, mNeptune, AsRed, mStrawberry and mPlum, as well as other fluorescent proteins in the green range derived from Aequorea Victoria.

B. Effector Moiety

An effector moiety in the delivery conjugate can be a molecule that is of essentially any chemical nature and possesses a desired function. Such function may include a biological activity for therapeutic purpose or for detection of a specific cell or tissue type.

1. Polypeptide Payloads

The delivery conjugate disclosed herein can include an effector or payload comprising a polypeptide. In some embodiments, the polypeptide payload includes, but is not limited to, a polypeptide, protein, glycoprotein, enzyme, hormone, antibody, antibody fragment, engineered immunoglobulin-like molecule, single chain antibody, conjugate, immune-costimulatory molecule, immunomodulatory molecule, fusion protein, protein scaffold, protein vaccine, growth factor, transcription factor, gene editing protein, ligand, antigen, cytokine, chemokine, tumor suppressor protein, toxin, e.g., endotoxin A, Colicin A, d-endotoxin, diphtheria toxin, Bacillus anthrox toxin, Cholera toxin, Pertussis toxin, Shiga toxin, or other bacterial protein toxin, fragments thereof, derivatives thereof, and variants thereof.

In some embodiments, the polypeptide payload contains a nuclear localization signal (NLS). Such nuclear localization signals are well known in the art. Non-limiting examples of a NLS include the following sequences: a monopartite NLS PKKKRKV (SEQ ID NO: 93), PAAKRVKLD (SEQ ID NO: 94) and K(K/R)X(K/R) (SEQ ID NO: 95), and a bipartite NLS KRPAATKKAGQAKKKK (SEQ ID NO: 96). Additional NLSs can be found in the NLS database described in Nair et al., Nucleic Acid Res, 2003, 1(31):397-9.

In certain embodiments, the polypeptide payload includes a targeting moiety or peptide that directs the payload to a target cell, a specific subcellular compartment, or a specific binding/interaction partner. In some embodiments, the peptide payload in a zinc finger TALE or CRISPR protein that can specifically bind to a polynucleotide sequence (genomic sequence) of interest. A payload can contain a specific tag. In some instances, a payload includes a specific polypeptide such as a peptide that interacts with a polypeptide, molecule, or compound on the surface of the cell to direct cell-specific entry of the delivery conjugate. In other instances, the payload includes a polypeptide that mediates, facilitates or directs intracellular trafficking of the delivery conjugate. For example, the polypeptide payload can contain a mitochondrial entry signal or a lysosomal entry signal.

2. Polynucleotide Payloads

The delivery conjugate disclosed herein can include an effector (cargo or payload) comprising a polynucleotide. A polynucleotide payload effector can include a nucleic acid, such as a oligonucleotide, cDNA, expression construct, mRNA, tRNA, small nuclear RNA (snRNA), ribosomal RNA (rRNA), short interfering RNA (siRNA), antisense RNA, interfering RNA, microRNA, and chimeric nucleic acid sequences. In some instances, the polynucleotide payload is single stranded, double stranded, or multistranded.

A polynucleotide payload includes, but is not limited to, a polynucleotide sequence encoding a polypeptide, protein, glycoprotein, enzyme, hormone, antibody, antibody fragment, engineered immunoglobulin-like molecule, single chain antibody, conjugate, immune-costimulatory molecule, immunomodulatory molecule, fusion protein, protein scaffold, protein vaccine, growth factor, transcription factor, gene editing protein, ligand, antigen, cytokine, chemokine, tumor suppressor protein, toxin, e.g., endotoxin A, Colicin A, d-endotoxin, diphtheria toxin, Bacillus anthrox toxin, Cholera toxin, Pertussis toxin, Shiga toxin, or other bacterial protein toxin, fragments thereof, derivatives thereof, and variants thereof.

C. Methods of Preparing Delivery Conjugates

Peptides of the present invention may be prepared using methods known in the art. For example, peptides may be produced by chemical synthesis, e.g., using solid phase techniques and/or automated peptide synthesizers, or by recombinant means. In certain instances, peptides may be synthesized using solid phase strategies on an automated multiple peptide synthesizer (Abimed AMS 422) using 9-fluorenylmethyloxycarbonyl (Fmoc) chemistry. The peptides can then be purified by reversed phase-HPLC and lyophilized. The peptides may alternatively be prepared by cleavage of a longer peptide or full-length protein sequence.

Peptides such as the cell penetrating peptide, solubilizing peptide, or configuring peptide can be synthesized in solution or on solid support in accordance with conventional techniques for generating synthetic peptides. Various automatic synthesizers are commercially available and can be used in accordance with known protocols. Short peptide sequences, usually from about 6 up to about 35 to 50 amino acids, can be readily synthesized by such methods. Alternatively, recombinant DNA techniques may be employed wherein a polynucleotide sequence which encodes a peptide disclosed herein is inserted into an expression vector, transformed or transfected into an appropriate host cell, and cultivated under conditions suitable for expression of the peptide.

In some embodiments, the delivery conjugate is generated by recombinant DNA techniques to produce a fusion polypeptide containing the cell penetrating peptide, solubilizing peptide, configuring peptide and polypeptide payload. Following recombinant expression of a delivery conjugate in a host cell or organism, the recombinant polypeptide can be isolated or purified by any technique known to those of skill in the art. These techniques involve, at one level, the homogenization and crude fractionation of the cells, tissue or organ to polypeptide and non-polypeptide fractions. The fusion polypeptide of interest may be further purified using chromatographic and electrophoretic techniques to achieve partial or complete purification (or purification to homogeneity). Analytical methods particularly suited to the preparation of a pure peptide are ion-exchange chromatography, gel exclusion chromatography, polyacrylamide gel electrophoresis, affinity chromatography, immunoaffinity chromatography and isoelectric focusing. Examples of efficient methods for purifying fusion polypeptides includes FPLC and HPLC.

A delivery conjugate to be used in a composition such as a therapeutic compositions can be isolated from other components such that the fusion polypeptide is purified to be free from the environment in which it was derived, e.g., a host cell. In some embodiments, the fusion polypeptide is subjected to fractionation to remove various components from the synthesis environment such that the resulting compositions substantially retain the biological activity of the fusion polypeptide. In some cases, the fusion polypeptide is a component of the composition that constitutes about 50%/c, about 60%, about 70%, about 80%, about 90%, about 95% or more of the polypeptides present in the composition. Various methods for quantifying the degree of purification of the polypeptides are known to those of skill in the art in light of the present disclosure. These include, for example, determining the specific activity of an active fraction, or assessing the amount of polypeptides within a fraction by SDS/PAGE analysis. In some embodiments, a method for assessing the purity of a fraction is to calculate the specific activity of the fraction, to compare it to the specific activity of the initial extract, and to thus calculate the degree of purity therein.

Methods for identifying a useful delivery conjugate include cell imaging techniques such as confocal laser scanning microscopy, electron microscopy, and time-lapse microscopy, and flow cytometry (FACS) to investigate the delivery of the conjugate into a cell, and tissue imaging such as immunohistochemistry and in vivo imaging. Useful devices for in vivo imaging of small animals include Maestro 2 Imager (PerkinElmer) and Pearl Imager (Li-Cor).

Provided herein are nucleic acids (polynucleotides) encoding conjugates described herein. Such polynucleotides can be constructed using standard recombinant DNA methodologies as described in, e.g., Sambrook et al., Molecular Cloning, A Laboratory Manual, Cold Springs Harbor Press (Cold Springs Harbor, N.Y. 1989). The polynucleotide encoding a delivery conjugate or constituent parts thereof, such as the cell penetrating peptide, solubilizing peptide, configuring peptide and polypeptide payload can be operably linked together to form an expression cassette. The expression cassette can be incorporated into a cloning or expression vector. A cloning vector can include, but is not limited to, one or more of the following: one or more marker genes, an enhancer element, a promoter, a transcription termination sequence and a signal sequence. The expression cassette can include a leader sequence to permit the recombinant expression of the delivery conjugate in a heterologous host cell.

The host cell may be any appropriate prokaryotic or eukaryotic cell, preferably it is well-defined bacteria, such as E. coli or yeast strain. Both such hosts are readily transformed and capable of rapid growth in fermentation cultures. In place of E. coli, other unicellular microrganisms can be employed, for instance, fungae and algae. In addition, other forms of bacteria such as salmonella or pneumococcus may be substituted for E. coli. Techniques for transforming recombinant plasmids in E. coli strains are widely known and described in, e.g., Sambrook et al., Molecular Cloning, A Laboratory Manual, Cold Springs Harbor Press (Cold Springs Harbor, N.Y. 1989).

D. Pharmaceutical Compositions and Administration

In some embodiments, a therapeutically effective amount of the conjugate or the composition is an amount sufficient for achieving a therapeutic benefit in the subject. In yet other embodiments, a therapeutically effective amount of the conjugate or the composition is an amount sufficient to target delivery of the therapeutic payload to a target cell or a target tissue.

The delivery conjugate disclosed herein can be formulated into a pharmaceutical composition for administration to a subject in need thereof. The pharmaceutical composition may comprise a pharmaceutically acceptable carrier. In certain aspects, pharmaceutically acceptable carriers are determined in part by the particular composition being administered, as well as by the particular method used to administer the composition. Accordingly, there is a wide variety of suitable formulations of pharmaceutical compositions of the present invention (see, e.g., REMINGTON'S PHARMACEUTICAL SCIENCES, 18TH ED., Mack Publishing Co., Easton, Pa. (1990)).

The pharmaceutical compositions of the invention are administered in a manner compatible with the dosage formulation, and in such amount as will be therapeutically effective or suitable for in vivo imaging. The quantity to be administered depends on a variety of factors including, e.g., the age, body weight, physical activity, and diet of the individual, the disease/disorder to be treated, and the stage or severity of the disease/disorder. In certain embodiments, the size of the dose may also be determined by the existence, nature, and extent of any adverse side effects that accompany the administration of a delivery conjugate in a particular individual. In general, the dose equivalent of a delivery conjugate or a pharmaceutical composition thereof is from about 1 ng/kg to about 500 mg/kg for a typical individual.

In the practice of this invention, the delivery conjugate or a pharmaceutical composition comprising the delivery conjugate can be administered, for example, intravenously, intracranially, intrathecally, intraspinally, intraperitoneally, intramuscularly, intralesionally, intranasally, subcutaneously, intracerebroventricularly, orally, topically, and/or by inhalation.

As used herein, the term “unit dosage form” refers to physically discrete units suitable as unitary dosages for humans and other mammals, each unit containing a predetermined quantity of a delivery conjugate calculated to produce the desired onset, tolerability, and/or therapeutic effects, in association with a suitable pharmaceutical excipient (e.g., an ampoule). In addition, more concentrated dosage forms may be prepared, from which the more dilute unit dosage forms may then be produced. The more concentrated dosage forms thus will contain substantially more than, e.g., at least 1, 2, 3, 4, 5, 6, 7, 8, 9, 10, or more times the amount of the delivery conjugate.

Methods for preparing such dosage forms are known to those skilled in the art (see, e.g., REMINGTON'S PHARMACEUTICAL SCIENCES, 18TH ED., Mack Publishing Co., Easton, Pa. (1990)). The dosage forms typically include a conventional pharmaceutical carrier or excipient and may additionally include other medicinal agents, carriers, adjuvants, diluents, tissue permeation enhancers, solubilizers, and the like. Appropriate excipients can be tailored to the particular dosage form and route of administration by methods well known in the art (see, e.g., REMINGTON'S PHARMACEUTICAL SCIENCES, supra).

Liquid dosage forms can be prepared by dissolving or dispersing a delivery conjugate and optionally one or more pharmaceutically acceptable adjuvants in a carrier such as, for example, aqueous saline (e.g., 0.9% w/v sodium chloride), aqueous dextrose, maltose, glycerol, ethanol, and the like, to form a solution or suspension, e.g., for oral, topical, or intravenous administration.

E. Therapeutic Applications

The delivery conjugate disclosed herein can be used to treat a subject having a disease involving a genetic loci that is imprinted, a loci that is dominant, or a loci that is haploinsufficient. Non-limiting disease associated with genomic imprinting include Prader-Willi syndrome, Angelman syndrome, Beckwith-Wiedemann syndrome, Rett syndrome, several types of tumors including Wilms' tumor, neuroblastoma, rhabdomyosarcoma, and lung cancer, a some neurological and psychiatric disorders. In some embodiments, the conjugate can be used to treat an autosomal dominant disease such as Huntington's disease, Kennedy's disease, another spinocerebellar disease, and an autosomal dominant disease of the eye, e.g., glaucoma and retinitis pigmentosa, Stargardt-like dominant macular dystrophy. Examples of diseases associated with haploinsufficiency that can be treated using the delivery conjugate include autism spectrum disorders, behavior disorders, Ehlers-Danlos syndrome (VEDS), osteogenesis imperfecta, Marfan syndrome, supravalvular aortic stenosis, Loeys-Dietz syndrome, 22q11.2 deletion syndrome, and Pitt-Hopkins syndrome. Additional diseases and disorders include depression, schizophrenia, HIV/AIDS, and cancers. In certain embodiments, the disease or disorder that can be treated or prevented using the delivery conjugate are associated with expression, overexpression, and/or activation of a specific gene or mutated gene. In some instances, the compositions and methods described herein can be used to treat Down's Syndrome. In other instances, the disease that can be treated is Alzheimer's disease.

### IV. Examples

The following examples are offered to illustrate, but not to limit, the claimed invention.

### Example 1: Subcutaneous Injection of Artificial Transcription Factor Restores Widespread Ube3a Expression in an Angelman Syndrome Mouse Brain

Angelman Syndrome (AS) is a neurologic disorder caused by loss of expression of the maternal copy of UBE3A in the brain (1,2). Due to brain-specific genetic imprinting at this locus, the paternal UBE3A is silenced by a long antisense transcript (3). Inhibition of the antisense transcript could lead to unsilencing of paternal UBE3A, thus providing a therapeutic approach to Angelman Syndrome. However, widespread delivery of gene regulators to the brain remains a difficult challenge. Here we report an engineered zinc finger-based artificial transcription factor containing a cell-penetrating peptide that was injected intraperitoneally (IP) or subcutaneously (SC), crossed the blood-brain barrier, increase Ube3a expression in the brain of an adult mouse model of Angelman Syndrome, and rescued phenotypic deficits in a balance and motor coordination. Unlike most viral vectors, the factor displayed widespread distribution throughout the brain. Ube3a was increased in both the hippocampus and cerebellum upon treatment by a factor that targeted a promoter of the antisense transcript, but not by a factor that had an altered DNA-binding domain. We believe this to be the first report of an engineered injectable gene-regulatory protein that can distribute widely in the brain and regulate the expression of an endogenous gene. Such an approach is useful for the treatment of Angelman Syndrome and other neurologic conditions.

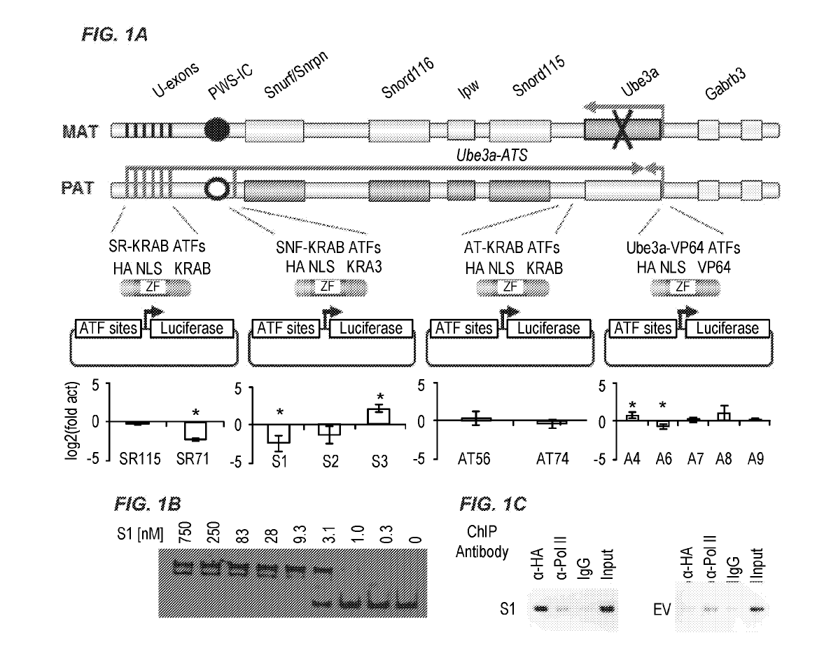

Angelman Syndrome (AS), a neurologic disorder of genetic origin that affects approximately 1:15,000 births, is characterized by intellectual disabilities, lack of speech, ataxia, and seizures (1, 2). The majority of AS cases is due to a 4-Mb de novo deletion of the maternal 15q11-q13 region resulting in the loss of UBE3A (ubiquitin-protein ligase E6-AP) expression in the brain (4,5). Due to brain-specific imprinting, the paternal allele is silenced, thus loss of the maternal allele causes UBE3A deficiency throughout the brain (6,7). Paternal silencing is caused by a long neuron-specific RNA transcript that overlaps and is antisense to Ube3a (Ube3a-ATS, FIG. 1A) (3). Inhibition of the extension of this transcript by insertion of a transcriptional termination sequence (8), topoisomerase inhibitor drugs (9), or antisense oligonucleotides (10) results in unsilencing of paternal Ube3a. These studies support the inhibition of the Ube3a-ATS as a strategy to enhance paternal Ube3a expression, and the possibility of postnatal phenotypic rescue.

We designed artificial transcription factors (ATFs) to repress expression of the Ube3a-ATS. ATFs are composed of a programmable DNA binding domain, such as zinc fingers, TALEs or CRISPR/Cas systems, and an effector domain that can activate or repress transcription (11, 12). Zinc finger-based ATFs have been evaluated in Phase 2 clinical trials (NCT00476931), which demonstrated that zinc finger ATFs could be well tolerated in human subjects. The lack of similar data for TALE and CRISPR ATFs led us to choose zinc fingers for our initial studies. In principle, activation of paternal Ube3a can be achieved either by super-activation of the Ube3a promoter or by repression of the inhibitory Ube3a-ATS transcript. Twelve zinc finger arrays consisting of six zinc finger modules each were constructed to 18-bp target sites, including five to a region upstream of the Ube3a locus, three upstream of the Snurf/Snrpn locus (thought to be an initiation site of the Ube3a-ATS)(13), two to sites common in the approximately nine upstream initiation sites of the Ube3a-ATS (3), and two to a region between the Snord115 cluster and Ube3a (FIG. 1A). To create simple artificial transcription factors, a VP64 transcriptional activation domain (14) was added to the five arrays that were designed to directly activate Ube3a, and a KRAB transcriptional repression domain (14) was added to the remaining arrays that were designed to repress the Ube3a-ATS. The ability of the factors to up- or down-regulate gene expression was assayed in HEK293T cells using luciferase reporter plasmids that harbored the target sites. The two factors that produced the most potent regulatory effect (>5-fold repression, p<0.05) were SR71 and S1, which had been designed to repress the Ube3a-ATS (FIG. 1A). The S1 zinc finger array was used for all subsequent experiments. An electromobility shift assay demonstrated that the S1 zinc finger array bound its target with an affinity of 3±1.5 nM (FIG. 1B). Chromatin immunoprecipitation followed by PCR (ChIP-PCR) demonstrated that the S1 factor was also able to bind its chromosomal target site in mouse Neuro2A cells (FIG. 1C).

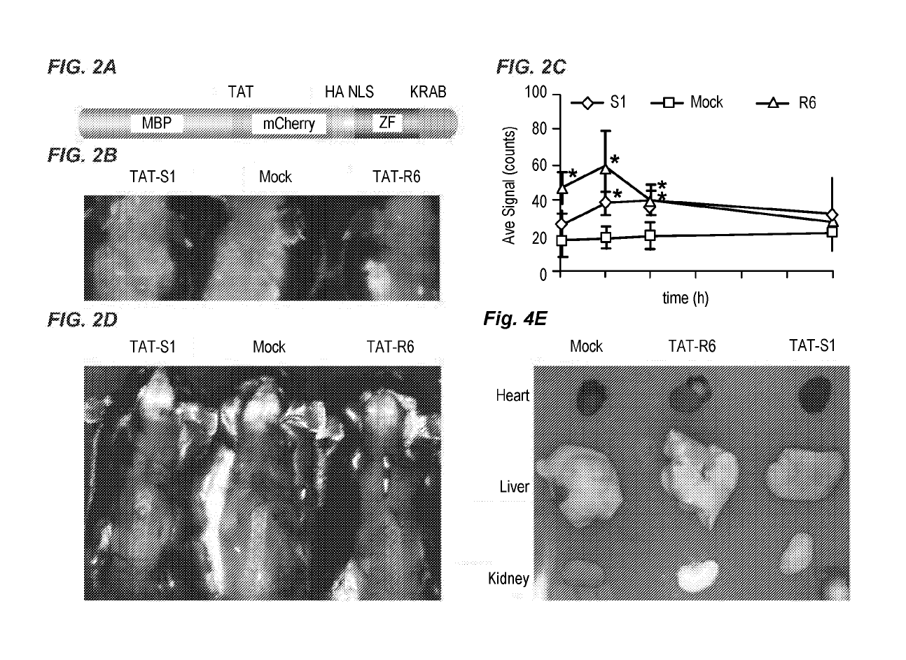

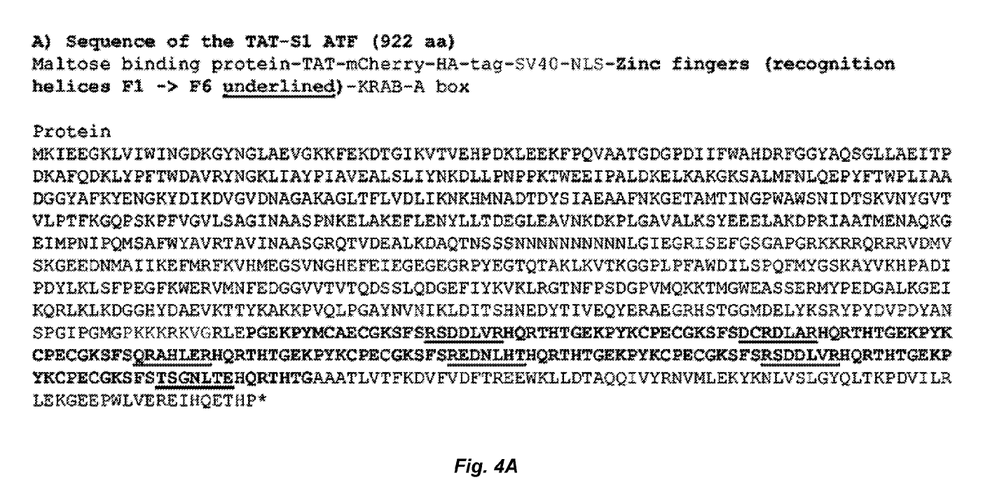

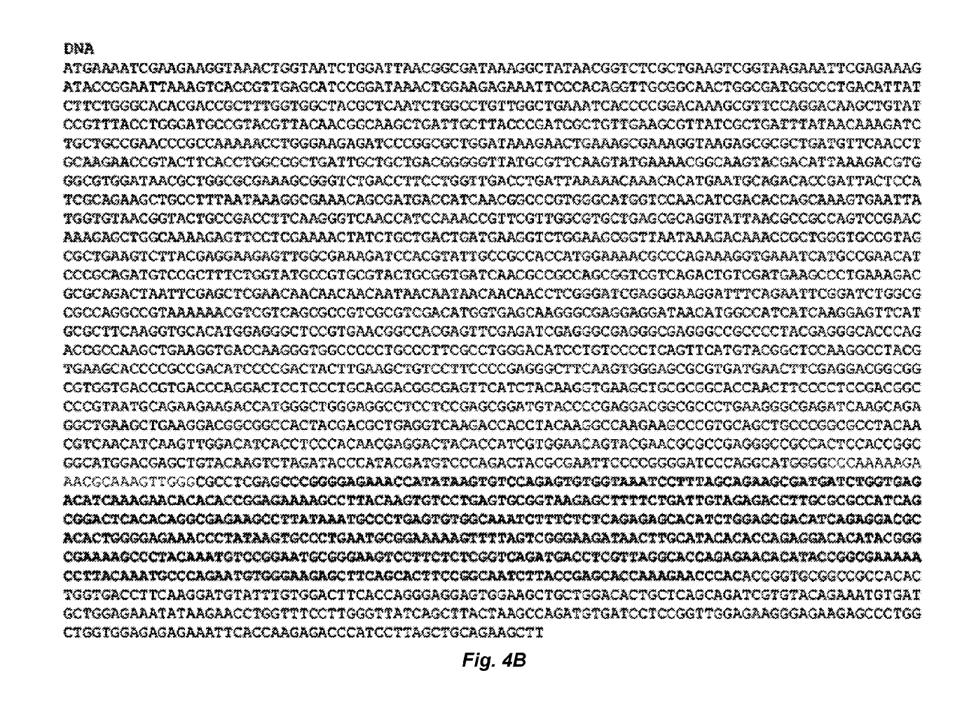

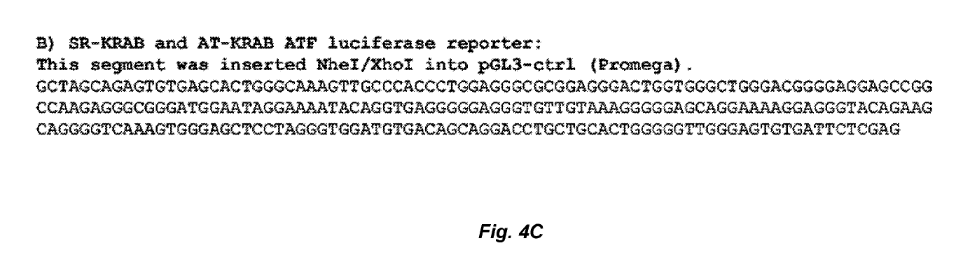

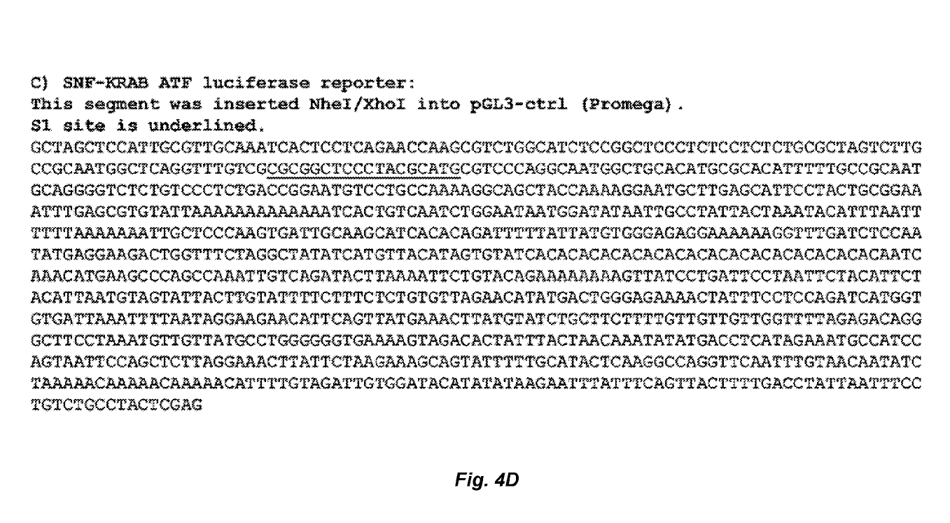

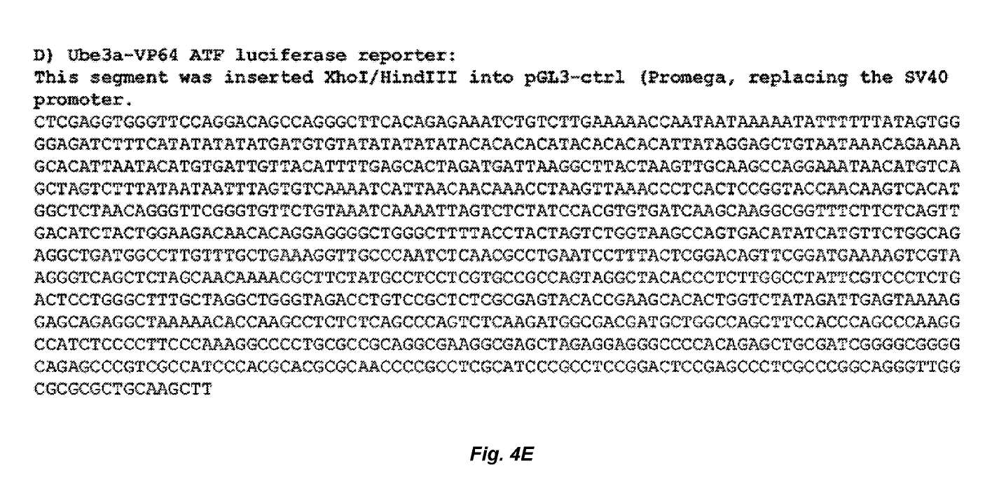

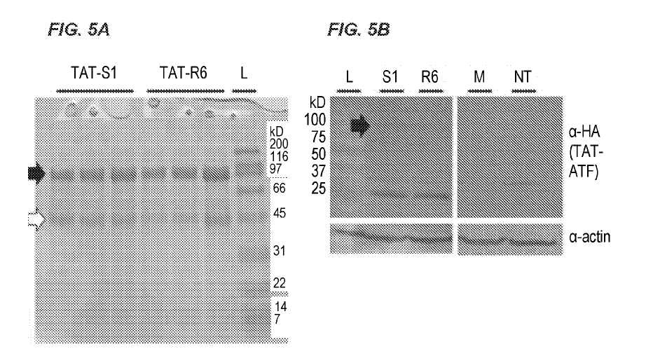

To address the need to achieve widespread activation of Ube3a in the brain, we constructed fusion proteins containing an N-terminal maltose binding protein for purification, the 10-aa transduction domain of the HIV-transactivator protein (TAT, residues 48-57) (15) as a cell-penetrating peptide, an mCherry red fluorescent protein to aid in protein solubility and visualization, an HA epitope tag for detection, and an SV40 nuclear localization signal to ensure nuclear delivery. The TAT cell-penetrating peptide had been previously shown to deliver other proteins to the brains of mice following intraperitoneal (IP) injection (16), thus providing a minimally invasive method for delivery of our ATFs. The C-terminus of the protein consisted of the S1 zinc finger array and KRAB domain (FIGS. 2A and 4). As a negative control, an identical construct was made with zinc finger array R6 that was not designed to bind at the Snurf/Snrpn locus (the closest match in the mouse genome contained two mismatches and was >30,000 bp from the nearest gene on chromosome 6). The TAT-S1 and TAT-R6 differ by only 26 aa in their DNA-binding domains. The 922-aa proteins were expressed in E. coli using a cold temperature induction (see Methods) and purified (FIG. 5A). Injection of adult wild type C57BL/6 mice (160-200 mg/kg, intraperitoneal [IP]) with purified TAT-S1 and TAT-R6 produced a peak of mCherry fluorescence in the brain within 4-8 hours (FIG. 2B-C). Full-length TAT-ATF protein could be detected by western blot analysis of brain nuclear lysates, in addition to lower molecular weight breakdown products (FIG. 5B). TAT-ATFs also distributed to other parts of the body (FIGS. 2D and 2E). These data suggest that TAT-ATFs injected IP can cross the blood brain barrier and enter nuclei in the cells of the brain.

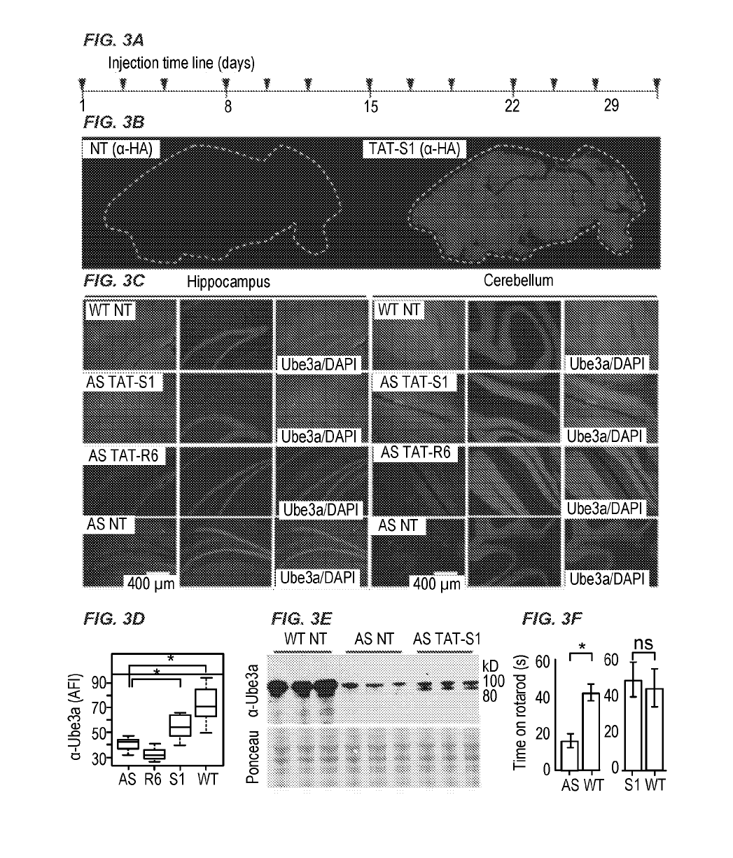

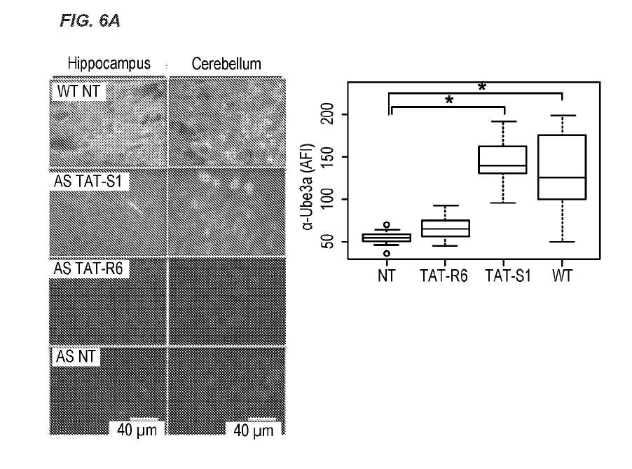

To investigate if TAT-S1 could perform its gene regulatory function in the brains of mice, we used a mouse model of AS¹⁷ that carried a transgene insertion in exon 5 at the 5′ end of the maternal Ube3a gene (AS mice, FIG. 1A). The insertion causes strong attenuation of Ube3a protein expression from the maternal allele. We expected our TAT-S1 to activate Ube3a protein expression from the paternal allele in this mouse model. However, given the half-life of our protein in the brain is only about 16 hours, and the KRAB effector domain is known to provide only transient transcriptional repression¹⁸, we reasoned that multiple administrations might ensure sufficient presence of the ATF over time to alter gene expression patterns. ATFs were injected (160-200 mg/kg, subcutaneous [SC]) three times per week for four weeks (FIG. 3A). A final injection was made four hours before harvest to examine distribution of the ATF. We observed no overt signs of toxicity during the four-week treatment period, based on normal appearance and behaviors of the mice and no visual organ pathology upon dissection (data not shown). We observed widespread distribution of ATFs throughout all regions of the brain (FIG. 3B). Ube3a protein was found to be increased in AS mice treated with TAT-S1, but not TAT-R6, in both hippocampus and cerebellum (p<0.01, n=3-4 mice, FIGS. 3C and 3D). The up-regulation of Ube3a was confirmed by western blot analysis of brain cytosolic lysates from three different mice that received TAT-S1 treatment (FIG. 3E). The treatment-dependent appearance of a particular Ube3a isoform can be clearly seen. Both the immunohistochemistry and western blot confirm that the treatment increased Ube3a to level a intermittent between no-treatment AS and wild-type control mice. An additional experiment in which the treatment was administered by IP injection for 7.5 weeks and visualized using a different antibody to Ube3a produced essentially similar results (FIG. 6A). AS mice displayed significant balance deficits compared to wild-type mice on a constant speed rotarod (p<7.6×10⁻⁵, n=16-19 mice, FIG. 3F, left).

However, there was no significant difference between wild type mice and AS mice treated for 7.5 weeks with TAT-S1 (FIG. 3F, right).

These studies demonstrate that purified TAT-S1 protein can be injected SC or IP in AS mice, cross the blood brain barrier, enter neurons throughout the brain, alter the expression of Ube3a, and rescue an AS phenotype. We believe this to be the first report of an engineered injectable gene-regulatory protein that can distribute widely in the brain and regulate the expression of an endogenous gene. Adult mice (≥2 months) were used for these experiments as it was expected that the Ube3a-ATS would be fully expressed from the paternal allele (19), as is the case for Angelman Syndrome diagnosis during early childhood. Distribution of ATFs was observed by in vivo mCherry fluorescence throughout the body of the mouse, including the cranium. Distribution in the brain was also shown by immunofluorescence based on a different part of the protein, the HA-tag, as well as by Ube3a increases in both the hippocampus and cerebellum, indicating that a portion of the protein containing at least HA-NLS-ZF-KRAB entered the nuclei of cells in the brain. Nuclear lysates from brain were shown by western blot to contain HA-tagged ATF protein.

Since the Ube3a-ATS is not expressed in glia cells, the observation that TAT-S1, but not TAT-R6, upregulated Ube3a indicates that the ATFs must have entered neurons. In addition, since TAT-S1 and TAT-R6 differ by only 26 as in their DNA-binding domains, the results also indicates that the function of TAT-S1 was dependent on the DNA-binding specificity of its zinc finger array, which was targeted and shown to bind upstream of the Snurf Snrpn locus. Therefore increased Ube3a expression was not caused by non-specific factors such as increased stress or immune response in the mouse, contaminants in the injection solution, or non-targeted actions by other parts of the protein such as the KRAB domain. TAT-R6 appeared to cause a slight decrease in Ube3a levels in the experiment shown in FIGS. 3A-3F, but a slight increase in the experiment shown in FIGS. 6A and 6B. These fluctuations are likely due to experimental variation using different cohorts on different days. We note that the presence of the MBP band in the injected material did not appear to influence the results, as both the TAT-S1 and TAT-R6 samples contained this band but only TAT-S1 increased Ube3a. Ube3a expression was not restored to full wild type levels, perhaps due to additional Ube3a-ATS transcripts initiating from upstream promoters. However, TAT-S1 treatment was sufficient to rescue the rotarod phenotype. The robust activation of Ube3a in the cerebellum upon TAT-S1 treatment may have contributed to our ability to rescue this particular phenotype.

The prospect for long-term treatment with proteins has been demonstrated by the clinical use of insulin and numerous other biologics (20). In principle, the use of ATF biologics with short-term effects similar to drugs may have advantages over approaches such as gene therapy, for which distribution, dosage and reversibility are problematic (11). ATFs designed to alter epigenetic information may provide more persistent effects (21,22). In addition, other programmable platforms such as TALE and CRISPR/Cas systems may be useful for gene regulation in the brain, even as purified protein (23,24). The development of such ATF methods could have significant impact for Angelman Syndrome and other neurologic disorders, since only small adjustments to the DNA-binding domain of the ATF might be required to target additional disease-related loci that are imprinted (Rett, Prader-Willi), dominant (Huntington), or haploinsufficient (22q11.2 deletion, Pitt-Hopkins, and many autism spectrum disease genes).

### Methods and Materials

In vitro transcription factor assays: Zinc finger coding regions were designed by modular assembly methods (26) and synthesized by BioBasic, Inc. (Ontario, Canada). The arrays were cloned using XhoI and NotI into the PGK promoter-driven mammalian expression vector pPGK-VP64 (29), which appended an N-terminal HA epitope tag and SV40 nuclear localization sequence, and either a C-terminal VP64 transcriptional activation domain (14) or a KRAB transcriptional repression domain, cloned at the NotI and PstI sites (14). Recognition helices of the zinc finger arrays are provided in Table 1. Target sites for the ATFs were cloned between NotI and XhoI sites upstream of the SV40 promoter in pGL3-control plasmids (Promega), as listed in FIGS. 4C-4E. In 24-well plates, HEK293T cells at 80% confluency in DMEM supplemented with 10⁰% fetal calf serum, 1 U/mL of penicillin and 1 μg/mL of streptomycin, were co-transfected with 100 ng of ATF expression plasmid, 25 ng of modified pGL3-control firefly luciferase reporter plasmid containing ATF target sites, and 25 ng of pRL-TK-Renilla Luciferase plasmid (as a transfection control, Promega), using Lipofectamine 2000 (Invitrogen). Cells were harvested 48 h post-transfection by removing media, washing with 500 μL of 1×DPBS (Life Technologies), followed by lysis in 100 μL of 1× Passive Lysis Buffer (Promega) with 1× Complete Protease Inhibitors (Roche). Clarified cell lysates (20 μL) were used to determine luciferase activity using DualGlo reagents (40 μL, Promega) in a Veritas microplate luminometer (Turner Biosystems). Luciferase signal was normalized to Renilla signal. All experiments were performed in duplicate and repeated on three different days.

Expression of TAT-S1 and TAT-R6 Protein: TAT-ATF coding regions were cloned into the pMAL-c2X vector (New England Biolabs) between EcoRI and HindIII. The full sequence of the TAT-S1 protein is provided in FIG. 4A, and the recognition helices of TAT-R6 are provided in Table 1. Proteins were expressed in NEB5α. E. coli (New England Biolabs) by inoculating 5 mL overnight cultures into 800 mL of Luria Broth medium (Sigma) with 50 μg/mL Carbenicillin. Cultures were shaken overnight at 37° C., then moved to 4° C. and induced with isopropyl β-D-1-thiogalactopyranoside (IPTG, 0.5 mM) and gently shaken for 48-72 hours. The cold temperature induction was critical to obtaining high yields; induction at room temperature or 37° C. was far less efficient. Culture was pelleted and re-suspended in 30 mL of Zinc Buffer A [ZBA; 10 mM Tris (pH 8.5), 90 mM KCl, 1 mM MgCl2, 100 μM ZnCl2]. Microfluidized lysates were run over columns of amylose resin (New England Biolabs, E8021L) that had been prepared by washing twice with deionized water, twice with Column Buffer [20 mM Tris-HCl (pH 7.4), 0.2 M NaCl, 1 mM EDTA], and twice with ZBA. Protein was eluted in Elution Buffer [ZBA, 500 mM maltose] and concentrated to ˜16 mg/mL using a Centricon Plus-70 (Millipore, UFC710008). Proteins were sterile filtered and routinely checked to assure no detectable endotoxins were present. Protein integrity and concentration was evaluated using Coomassie-stained gels and concentration established via Nanodrop A280 UV absorption. Protein concentrations for injections refer only the concentration of the 100 kD full-length protein band, which was considered to be half of the full-length+MBP band intensities (FIG. 5A). Proteins were stored at −80° C. in Injection Buffer [Elution Buffer, 30% glycerol, 4 mM dithiothreitol (DTT)].

Electromobility shift assays (EMSA): Biotin-labeled DNA targets were generated by PCR-amplification using a 5′ biotinylated forward primer of 69-mer oligonucleotides containing 18-base pair zinc finger target site (Table 2). PCR reactions contained unlabeled reverse primer in a 4:1 ratio over the biotinylated primer. Amplified targets were column purified (Qiagen). Protein-DNA complexes were mixed on ice, and then incubated in the dark for 1.5 h at room temperature in ZBA, 0.05% NP-40, 0.1 mg/mL BSA, 100/o glycerol, and 35-75 pM target oligo. Protein-DNA complexes were separated from the unbound probe using 7% polyacrylamide TBE gels run in 0.5×TBE (Bio-Rad), and then transferred onto Biodyne B nylon membranes. Complexes were UV cross-linked to the membranes for 4 min (UV Stratalinker 1800, Stratagene). The biotinylated DNA was visualized using the LightShift Chemiluminescent EMSA Kit (Pierce) according to the manufacturer's protocol. Equilibrium binding constants (apparent KD) were calculated from protein titration experiments. Gel images on X-ray film (Denville Scientific) were scanned and then quantitated using ImageJ. All reported EMSA measurements were averages of at least three experiments performed with independent protein dilutions. Representative data is shown in FIG. 1B.

ChIP-PCR assay: The mouse neuroblastoma cell line Neuro-2a (ATCC #CCL-131) was grown in Dulbecco's modified Eagle's medium (DMEM) supplemented with 10% bovine calf serum (BCS). Neuro-2a cells were grown to 70% confluency and transfected with 15 μg S1-KRAB expressing plasmid per 10-cm dish using Lipofectamine 2000 (Life Technologies). Cells were cross-linked 48 h post transfection by incubation with 1% formaldehyde solution for 10 min at room temperature. ChIP assays were performed as previously described with minor modifications (27). 30 μg of sonicated chromatin was incubated with 3 μg of monoclonal anti-HA antibody (Covance HA. 11 clone 16B12), monoclonal anti-RNA Polymerase II antibody (Covance 8WG16) or control mouse IgG (SIGMA). Immunoprecipitates were captured using 3 μg rabbit anti-mouse serum and StaphA cells (Sigma-Aldrich). After washes and reversal of DNA-RNA-protein cross-links, standard PCR was performed to confirm specific binding to the S1 target sequence in ChIP samples as compared to 0.1% input control. All primer sequences are listed in Table 2. Primers Snurf-F and Snurf-R to the mouse Snurf gene promoter target site were used as positive control primers, while mmchr4-F and mmchr4-R to a region on mouse chromosome 4 served as a negative control. Specific fragments were amplified using GoTaq (Promega) DNA polymerase (2 min at 95° C.; 30 sec at 95° C., 30 s at 60° C., 30 s at 72° C., 35 cycles; 5 min at 72° C.). PCR products were separated on a 1.5% agarose gel and visualized using the Gel Doc™ XR+ System (BioRad).

Mice: All mice were maintained and experiments conducted according to University of California, Davis approved Institutional Animal Care and Use Committee guidelines and protocols. The AS mouse model (17) was obtained from Jackson Laboratories (Stock 004477, 129-Ube3a^{tm1Alb/}J) and maintained on a mixed background of SV129 and C57BL′6. Wild type C57BL/6J mice (Jackson Laboratories) and SV129 mice (Charles River Laboratories) were used for matings with Ube3a-deficient female mice. Genotyping was performed according to the supplier's protocol, with the modification that the annealing temperature was changed to 60° C. Primers are listed in Table 2. All animals receiving injections were older than two months of age at the time of injection.

Maestro™ in-vivo imaging of mice: All mice used in this study were C57BL/6 male mice. All mouse images were performed on a Maestro™ 2 Imager (PerkinElmer), use and training provided by the Center for Molecular and Genomic Imaging at UC Davis. The green filter was used with acquisition settings of 550-800 nm in 10 nm steps. Before injections, an image was taken of the purified protein using the auto-expose option. Timepoints used were 15 min, 4 h, 8 h and 24 h post injection. At each timepoint, fluorescent images were taken of the injection site, the head and the back. Exposure times for the injection site, head, and back images were established from a previous pilot experiment at 229 ms, 1959 ms and 1986 ms, respectively. On all images, a mock animal served as a control. All images were based on the same raw inputs for mCherry signal and background mouse signal. Analysis of mCherry signal was performed by measuring the mCherry signal in each mouse forebrain area using Maestro™ software. The measurement area was the same for each mouse across all treatments.

Immunohistochemistry (IHC): For the images in FIGS. 3B and 3C (50 μm thick sections) one brain hemisphere was washed in 1×TBS [50 mM Tris-Cl, pH 7.5, 150 mM NaCl], fixed in 10% buffered paraformaldehyde overnight, then placed into 30% sucrose for 3 days. Following brain saturation, the tissue was frozen in Tissue-Tek™ CRYO-OCT Compound (Thermo-Fisher 14-373-65), then sectioned on a Leica cryotome. Blocking was performed using Superblock (Life Technologies) for 1 h, followed by a 10% goat serum, 0.3% Triton X in 1× TBS for 2 h. Following aspiration, slides were stained with primary anti-HA 1:150 (Roche 12013819001 that was directly labeled with an Apex Antibody labeling kit (Life Technologies, A10470), or anti-Ube3a 1:100 (Sigma E8655) labeled with the Apex Antibody labeling kit, in 5% goat serum, 0.15% Triton X and incubated at 4° C. overnight. Slides were washed three times with 1×TBS, stained with 4′,6-diamidino-2-phenylindole (DAPI) for 10 min, washed three times with 1×TBS, and then mounted with coverslip using Prolong Gold (Life Technologies). Images were acquired using a Leica DM6000B epifluorescent microscope. A 10% linear reduction in brightness was applied equally to the green channel of all images to reduce autoflorescence and clarify features. All fluorescence intensity measurements were performed on unaltered images using ImageJ software 1.48 v. Fluorescence intensities were measured in the hilus region of the dentate gyrus in the hippocampus and the Purkinje cell layer in the cerebellum.

For the images in FIGS. 6A and 6B (5 μm sections) paraffin embedding was utilized.

One hemisphere of the brain was placed in 1×TBS, and then fixed in 4% paraformaldehyde overnight. Brains were fixed in an additional 3 h in 10% paraformaldehyde solution, and then placed into wax for sectioning. Slides of these sectioned tissues were incubated overnight at 56° C., washed with xylene twice for 10 min, and then soaked in 100% ethanol twice for 10 min. Antigen retrieval was performed using 1× Target Retrieval Solution (Dako) at 95° C. for 1 h, then washed in 1×SSC [150 mM NaCl, 15 mM Sodium Citrate] at room temperature for 5 min while rocking gently. Blocking was performed using Superblock for 1 h, followed by a 10% goat serum and 0.3% Triton X in 1×TBS for 2 h. Following aspiration, slides were stained with primary anti-Ube3a 1:1000 (Atlas HPA039410) with coverslip and incubated at 4° C. overnight. Coverslip were removed and the slide was washed three times with 1×TBS. The secondary antibody, goat-anti-rat Alexa Flouro 647 1:1000 (Abcam 150159) or goat-anti-rabbit Alexa Flouro 488 1:1000 (Abcam 150077) in 1×TBS, 5% goat serum and 0.15% Triton, was applied for 2 h, washed three times with 1×TBS, and then dried. Coverslips were applied with DAPI-containing Vectashield (Vector Laboratories, H-1200). The images in FIGS. 6A and 6B received no alterations. All fluorescence intensity measurements were performed on unaltered images as above.

Western blot: For the experiments in FIG. 3E, mouse brains were harvested and immediately snap frozen at −80° C. Proteins were extracted using the CelLytic™ NuCLEAR™ Extraction Kit (Sigma-Aldrich) according to the manufacturer's instructions. Protein concentration was determined utilizing the BCA Protein Assay kit (Pierce). 20 μg of protein were separated on a 10-20% Novex Tris-Glycine Gel (Life Technologies) using MOPS buffer. Proteins were transferred onto nitrocellulose membranes using the Mini Trans-Blot® Cell (BioRad) and even protein loading was evaluated using PonceauS staining. Membranes were then blocked with 5% dry milk in TBST (20 mM Tris, pH 7.5, 150 mM NaCl, 0.1% Tween) and probed with anti-Ube3a antibody (Sigma, E8655, 1:1000 dilution). After incubation with goat anti-mouse HRP (Santa Cruz, SC2055, 1:2500 dilution), chemiluminescence was detected using the ECL Prime Western Blotting Detection Reagent (Amersham, RPN2232).

For the experiments in FIGS. 5A and 5B, one hemisphere from each brain was flash frozen in liquid nitrogen. Tissues were treated first with hypotonic buffer [2 mM Hepes, 1 mM KCl, 0.1 mM EDTA, 10% Glycerol, 1× protease inhibitor cocktail (Roche, 11873580001), 50 μM PMSF, 50 ul of 100 μM DTT, in DEPC treated water], and then ground with motorized pestle for 35 s. The remaining nuclear pellet after centrifugation was lysed with RIPA buffer [50 mM Tris-Cl, pH 7.4, 150 mM NaCl, 0.25% NaDeoxycholate, 1% NP-40 (IGEPAL), 1 mM EDTA, 1× Protease Inhibitor Cocktail]. The nuclear lysate was quantified using a BCA assay (Life Technologies). Polyacrylamide gels (10-20% Tris Glycine, Biorad) were loaded with 250 μg of protein per sample and run at 115 V for 1.5 h, and then transferred to PVDF membrane using a semi-dry blotting apparatus at 75 V for 1.5 h. The membrane was blocked in 5% dry milk in TBST [20 mM Tris, pH 7.5, 150 mM NaCl, 0.1% Tween], and then probed for 48 h at 4° C. with primary a rat anti-HA antibody conjugated to HRP at 1:100-250 (Roche, 12013819001). The western was incubated with the ECL Plus Reagent (Amersham, RPN2133) for 15 min and imaged on a Storm 860 imager (Molecular Dynamics). Images were not altered.

Rotarod Testing: A constant speed rotarod (Rota-rod 7600, Ugo Basile Biological Research Apparatus) was used to measure balance and motor coordination. Training consisted of three 1-min sessions at least 10 min apart for three days using a speed of 16 r.p.m. During training, the mouse was replaced on the rod when it fell. Testing on the fourth day consisted of a single 1-min trial using 24 r.p.m. During testing the trial terminated when the mouse fell. AS mice receiving TAT-S1 treatment were injected over 7.5 weeks as in FIGS. 6A and 6B. The rotarod assay was conducted during the final week of injections. Mock-injected wild-type mice received ATF Injection Buffer.

Although the foregoing invention has been described in some detail by way of illustration and example for purposes of clarity of understanding, one of skill in the art will appreciate that certain changes and modifications may be practiced within the scope of the appended claims. All patents, patent applications, and other publications, including GenBank Accession Numbers, cited in this application are incorporated by reference in the entirety for all purposes.

### REFERENCES

i. Lalande, M. & Calciano, M. A. Molecular epigenetics of Angelman syndrome. Cell Mol Life Sci 64, 947-960 (2007).
2. Angelman, H. ‘Puppet’ children. A report on three cases. Dev Med Child Neurol 7, 681-688 (1965).
3. Landers, M. et al. Regulation of the large (approximately 1000 kb) imprinted murine Ube3a antisense transcript by alternative exons upstream of Snurf/Snrpn. Nucleic Acids Res 32, 3480-3492 (2004).
4. Kishino, T., Lalande, M. & Wagstaff, J. UBE3A/E6-AP mutations cause Angelman syndrome. Nat Genet 15, 70-73 (1997).
5. Chamberlain, S. J. et al. Induced pluripotent stem cell models of the genomic imprinting disorders Angelman and Prader-Willi syndromes. Proc Natl Acad Sci USA 107, 17668-17673, (2010).
6. Albrecht, U. et al. Imprinted expression of the murine Angelman syndrome gene, Ube3a, in hippocampal and Purkinje neurons. Nat Genet 17, 75-78 (1997).
7. Gustin, R M. et al. Tissue-specific variation of Ube3a protein expression in rodents and in a mouse model of Angelman syndrome. Neurobiology of disease 39, 283-291, (2010).
8. Meng, L. et al. Truncation of Ube3a-ATS unsilences paternal Ube3a and ameliorates behavioral defects in the Angelman syndrome mouse model. PLoS genetics 9, e1004039, (2013).
9. Huang, H. S. et al. Topoisomerase inhibitors unsilence the dormant allele of Ube3a in neurons. Nature 481, 185-189, (2012).
10. Meng, L. et al. Towards a therapy for Angelman syndrome by targeting a long non-coding RNA. Nature 518, 409-412, (2015).
11. Bailus, B. J. & Segal, D. J. The prospect of molecular therapy for Angelman syndrome and other monogenic neurologic disorders. BMC neuroscience 15, 76, (2014).
12. Blancafort, P. & Beltran, A. S. Rational Design, Selection and Specificity of Artificial Transcription Factors (ATFs): The Influence of Chromatin in Target Gene Regulation. Comb Chem High Throughput Screen 11, 146-158 (2008).
13. Bressler, J. et al. The SNRPN promoter is not required for genomic imprinting of the Prader-Willi/Angelman domain in mice. Nat Genet 28, 232-240, (2001).
14. Beerli, R R, Segal, D. J., Dreier, B. & Barbas III, C. F. Toward controlling gene expression at will: specific regulation of the erbB-2/HER-2 promoter by using polydactyl zinc finger proteins constructed from modular building blocks. Proc Natl Acad Sci USA 95, 14628-14633 (1998).
15. Vives, E., Brodin, P. & Lebleu, B. A truncated HIV-1 Tat protein basic domain rapidly translocates through the plasma membrane and accumulates in the cell nucleus. J Biol Chem 272, 16010-16017 (1997).
16. Gong, B. et al. Ubiquitin hydrolase Uch-L1 rescues beta-amyloid-induced decreases in synaptic function and contextual memory. Cell 126, 775-788, (2006).
17. Jiang, Y. H. et al. Mutation of the Angelman ubiquitin ligase in mice causes increased cytoplasmic p53 and deficits of contextual learning and long-term potentiation. Neuron 21, 799-811 (1998).
18. Gilbert, L. A. et al. Genome-Scale CRISPR-Mediated Control of Gene Repression and Activation. Cell 159, 647-661, (2014).
19. Leung, K. N., Vallero, R. O., DuBose, A. J., Resnick, J. L. & LaSalle, J. M. Imprinting regulates mammalian snoRNA-encoding chromatin decondensation and neuronal nucleolar size.

Hum Mol Genet 18, 4227-4238, (2009).
20. Espiritu, M. J., Collier, A. C. & Bingham, J. P. A 21st-century approach to age-old problems: the ascension of biologics in clinical therapeutics. Drug Discov Today 19, (2014).
21. Falahi, F., Sgro, A. & Blancafort, P. Epigenome engineering in cancer: fairytale or a realistic path to the clinic? Front Oncol 5, 22, (2015).
22. Jurkowski, T. P., Ravichandran, M. & Stepper, P. Synthetic epigenetics-towards intelligent control of epigenetic states and cell identity. Clin Epigenetics 7, 18, (2015).
23. Zuris, J. A. et al. Cationic lipid-mediated delivery of proteins enables efficient protein-based genome editing in vitro and in vivo. Nat Biotechnol 33, 73-80, (2015).
24. Liu, J., Gaj, T., Patterson, J. T., Sirk, S. J. & Barbas, C. F., 3rd. Cell-penetrating peptide-mediated delivery of TALEN proteins via bioconjugation for genome engineering. PloS one 9, e85755, (2014).
25. Bhakta, M. S. & Segal, D. J. The generation of zinc finger proteins by modular assembly.

Methods Mol Biol 649, 3-30, (2010).
26. Szczepek, M. et al. Structure-based redesign of the dimerization interface reduces the toxicity of zinc-finger nucleases. Nat Biotechnol 25, 786-793 (2007).
27. O'Geen, H., Frietze, S. & Farnham, P. J. Using ChIP-seq technology to identify targets of zinc finger transcription factors. Methods Mol Biol 649, 437-455, (2010).

TABLE 1
DNA binding sequences (5′→3′) and recognition helices (positions -1, 1,
2, 3, 4, 5, & 6 for fingers F6→F1 of the zinc finger arrays used in this
study.

| ZF Name | F6 | F5 | F4 | F3 | F2 | F1 |
|---|---|---|---|---|---|---|
| A4 | CCA | GAC | TAG | TAG | GTA | AAA |
| helices | TSHSLTE | DPGNLVR | REDNLHT | REDNLHT | QSSSLVR | QRANLRA |
| | (SEQ ID | (SEQ ID | (SEQ ID | (SEQ ID | (SEQ ID | (SEQ ID |
| | NO: 1) | NO: 2) | NO: 3) | NO: 4 | NO: 5) | NO: 6) |
| A6 | GGC | CAA | GAG | GGT | GTA | GCC |
| helices | DPGHLVR | QSGNLTE | RSDNLVR | TSGHLVR | QSSSLVR | DCRDLAR |
| | (SEQ ID | (SEQ ID | (SEQ ID | (SEQ ID | (SEQ ID | (SEQ ID |
| | NO: 7) | NO: 8) | NO: 9) | NO: 10) | NO: 11) | NO: 12) |
| A7 | ATA | GAT | TGA | GTA | AAA | GGA |
| helices | QKSSLIA | TSGNLVR | QAGHLAS | QSSSLVR | QRANLRA | QRAHLER |
| | (SEQ ID | (SEQ ID | (SEQ ID | (SEQ ID | (SEQ ID | (SEQ ID |
| | NO: 13) | NO: 14) | NO: 15) | NO: 16) | NO: 17) | NO: 18) |
| A8 | GCC | GCA | GGC | GAA | GGC | GAG |
| helices | DCRDLAR | QSGDLRR | DPGHLVR | QSSNLVR | DPGHLVR | RSDNLVR |
| | (SEQ ID | (SEQ ID | (SEQ ID | (SEQ ID | (SEQ ID | (SEQ ID |
| | NO: 19) | NO: 20) | NO: 21) | NO: 22) | NO: 23) | NO: 24) |
| A9 | AGG | GCT | CGG | AGT | CCG | GAG |
| helices | RSDHLTN | TSGELVR | RSDKLTE | HRTTLTN | RNDTLTE | RSDNLVR |
| | (SEQ ID | (SEQ ID | (SEQ ID | (SEQ ID | SEQ ID | (SEQ ID |
| | NO: 25) | NO: 26) | NO: 27) | NO: 28) | NO: 29) | NO: 30) |
| AT56 | GGT | GAG | GGG | GAG | GGT | GTT |
| helices | TSGHLVR | RSDNLVR | RSDKLVR | RSDNLVR | TSGHLVR | TSGSLVR |
| | (SEQ ID | (SEQ ID | (SEQ ID | (SEQ ID | (SEQ ID | (SEQ ID |
| | NO: 31) | NO: 32) | NO: 33) | NO: 34) | NO: 35) | NO: 36) |
| AT74 | ATG | GAA | TAG | GAA | AAT | ACA |
| helices | RRDELNV | QSSNLVR | REDNLHT | QSSNLVR | TTGNLTV | SPADLTR |
| | (SEQ ID | (SEQ ID | (SEQ ID | (SEQ ID | (SEQ ID | (SEQ ID |
| | NO: 373 | NO: 38) | NO: 39) | NO: 40) | NO: 41) | NO: 42) |
| S1 | CAT | GCG | TAG | GGA | GCC | GCG |
| helices | TSGNLTE | RSDDLVR | REDNLHT | QRAHLER | DCRDLAR | RSDDLVR |
| | (SEQ ID | (SEQ ID | (SEQ ID | (SEQ ID | (SEQ ID | (SEQ ID |
| | NO: 43) | NO: 44) | NO: 45) | NO: 46) | NO: 47) | NO: 48) |
| S2 | GCA | ATG | GCT | GCA | CAT | GCG |
| helices | QSGDLRR | RRDELNV | TSGELVR | QSGDLRR | TSGNLTE | RSDDLVR |
| | (SEQ ID | (SEQ ID | (SEQ ID | (SEQ ID | (SEQ ID | (SEQ ID |
| | NO: 49) | NO: 50) | NO: 51) | NO: 52) | NO: 53) | NO: 54) |
| S3 | GAT | CTG | GAG | GAA | ATA | GTT |
| helices | TSGNLVR | RNDALTE | RSDNLVR | QSSNLVR | QKSSLIA | TSGSLVR |
| | (SEQ ID | (SEQ ID | (SEQ ID | (SEQ ID | (SEQ ID | (SEQ ID |
| | NO: 55) | NO: 56) | NO: 57) | NO: 58) | NO: 59) | NO: 60) |
| SR71 | GCA | GGA | CCT | GCT | GCA | CTG |
| helices | QSGDLRR | QRAHLER | TKNSLTE | TSGELVR | QSGDLRR | RNDALTE |
| | (SEQ ID | (SEQ ID | (SEQ ID | (SEQ ID | (SEQ ID | (SEQ ID |
| | NO: 61) | NO: 62) | NO: 63) | NO: 64) | NO: 65) | NO: 66) |
| SR115 | CCT | AGG | GTG | GAT | GTG | ACA |
| helices | TKNSLTE | RSDHLTN | RSDELVR | TSGNLVR | RSDELVR | SPADLTR |
| | (SEQ ID | (SEQ ID | (SEQ ID | (SEQ ID | (SEQ ID | (SEQ ID |
| | NO: 67) | NO: 68) | NO: 69) | NO: 70) | NO: 71) | NO: 72) |
| R6 (neg ctrl) | AAA | GTT | GCC | CAC | CCT | GGA |
| helices | QRANLRA | TSGSLVR | DCRDLAR | SKKALTE | TKNSLTE | QRAHLER |
| | (SEQ ID | (SEQ ID | (SEQ ID | (SEQ ID | (SEQ ID | (SEQ ID |
| | NO: 73) | NO: 74) | NO: 75) | NO :76) | NO: 77) | NO: 78) |

TABLE 2
Printers used in the study.

| Name | Sequence |
|---|---|
| | Primers for ChIP-PCR |
| Snurf-F | 5′-CTCTCCTCTCTGCGCTAGTC-3′ (SEQ ID NO: 79) |
| Snurf-R | 5′-AGAGACCCCTGCATTGCG-3′ (SEQ ID NO: 80) |
| mmchr4-F | 5′-GAGCTATGGCCCATTGATGT-3′ (SEQ ID NO: 81) |
| mmchr4-R | 5′-AATAGTGGGATGGTGGGAGA-3′ (SEQ ID NO: 82) |
| | Sequencing primers for Luciferase plasmids |
| RVprimer3-f | 5′-CTAGCAAATAGGCTGTCC-3′ (SEQ ID NO: 83) |
| GLprimer2-r | 5′-CTTATGTTTTTGGCGTCTTCC-3 (SEQ ID NO: 84) |
| | EMSA of purified ATFs |
| Biotinylated f | /5Biosg/CCTCTTCGCTATTACGCCAGC-3′ (SEQ ID NO: 85) |
| Primer r | 5′-CACCCTGACTCGAGTACGATCGAACGTTC-3′ (SEQ ID |
| | NO: 86) |
| S1 target site | 5′-CCTCTTCGCTATTACGCCAGC |
| | CATGCGTAGGGAGCCGCG |
| | GAACGTTCGATCGTACTCGAGTCAGGGTG-3′ (SEQ ID NO: 87) |
| | AS mice genotyping primers |
| R1965 | 5′-GCTCAAGGTTGTATGCCTTGGTGCT-3′ (SEQ ID NO: 88) |
| WTF1966 | 5′-AGTTCTCAAGGTAAGCTGAGCTTGC-3′ (SEQ ID NO: 89) |
| ASF1967 | 5′-TGCATCGCATTGTCTGAGTAGGTGTC-3′ (SEQ ID NO: 90) |

What is claimed is:

1. A delivery conjugate comprising (1) a cell penetrating peptide: (2) a nuclear localization signal; and (3) an effector.

2. The conjugate of claim 1, further comprising an epitope tag.

3. The conjugate of claim 1 or 2, further comprising a solubilizing peptide and a configuring peptide.

4. The conjugate of claim 1, wherein the cell penetrating peptide is selected from a group consisting of a HIV TAT protein or a fragment thereof comprising the protein transduction domain, Drosophila Antennapedia (Antp) peptide and polyarginine (Arg₈) peptide.

5. The conjugate of claim 3, wherein the solubilizing peptide is a maltose-binding protein (MBP) peptide.

6. The conjugate of claim 3, wherein the configuring peptide is a fluorophore.

7. The conjugate of claim 2, wherein the epitope tag is an HA epitope tag.

8. The conjugate of claim 1, wherein the effector is a polypeptide.

9. The conjugate of claim 8, which is a fusion protein wherein (1), (2), and (3) are linked by peptide bonds.

10. The conjugate of claim 1, wherein the nuclear localization signal (NLS) is derived from SV40.

11. The conjugate of claim 1, comprising from the N-terminus an HIV TAT protein or a fragment thereof comprising the protein transducing domain; fluorephore mCherry; HA epitope tag; SV40 SNL; and a polypeptide effector.

12. The conjugate of claim 1, wherein the effector is a polynucleotide.

13. The conjugate of claim 1, further comprising a cell targeting signal.

14. A method of delivering an effector into a subject in need thereof, the method comprising administering to the subject the delivery conjugate of claim 1.

15. The method of claim 14, wherein the subject is a human subject.

16. The method of claim 15, wherein the human subject suffers from a disease involving a genetic locus that is imprinted, dominant, or haploinsufficient or a disease which can be treated by in activation or repression of a gene.

17. The method of claim 16, wherein the disease is selected from the group consisting of Angelman Syndrome, Prader-Willi Syndrome, Rett Syndrome, Huntington Disease, 22q11.2 deletion Syndrome, Pitt-Hopkins Syndrome, autism spectrum disorder, depression, schizophrenia, and HIV/AIDS.

18. The method of claim 14, wherein the administering comprises administering intraperitoneally, subcutaneously, intravenously, or intracranially.

19. A polynucleotide sequence encoding the delivery conjugate of claim 9.

20. The polynucleotide sequence of claim 19, wherein the conjugate further comprises an epitope tag.

21. The polynucleotide sequence of claim 19 or 20, wherein the conjugate further comprises a solubilizing peptide and a configuring peptide.

22. The polynucleotide sequence of claim 19, wherein the cell penetrating peptide is selected from a group consisting of a HIV TAT protein or a fragment thereof comprising the protein transduction domain, Drosophila Antennapedia (Antp) peptide and polyarginine (Arg8) peptide.

23. The polynucleotide sequence of claim 21, wherein the solubilizing peptide is a MBP peptide.

24. The polynucleotide sequence of claim 21, wherein the configuring peptide is a fluorophore.

25. The polynucleotide sequence of claim 19, wherein the epitope tag is an HA epitope tag.

26. The polynucleotide sequence of claim 19, wherein the nuclear localization signal (NLS) is derived from SV40.

27. The polynucleotide sequence of claim 19, wherein the conjugate comprises from the N-terminus an HIV TAT protein or a fragment thereof comprising the protein transducing domain; fluorephore mCherry; HA epitope tag; SV40 SNL; and a polypeptide effector.

28. An expression cassette comprising a promoter operably linked to the polynucleotide sequence of claim 19 or 27.

29. A host cell comprising the expression cassette of claim 28.
